(19) United States
(12) Patent Application Publication
(10) Pub. No.: US 2011/0117607 A1
(43) Pub. Date: May 19, 2011

(54) ANNULAR COMPRESSION SYSTEMS AND METHODS FOR SAMPLE PROCESSING DEVICES

(75) Inventors: William Bedingham, Woodbury, MN (US); Peter D. Ludowise, Cottage Grove, MN (US); Jeffrey C. Pederson, Minneapolis, MN (US); Barry W. Robole, Woodville, WI (US); Christopher R. Kokaisel, St. Paul, MN (US)

(73) Assignee: 3M Innovative Properties Company

(21) Appl. No.: 12/617,905

(22) Filed: Nov. 13, 2009

(51) Int. Cl. C12P 19/34; C12M 1/00

(52) U.S. Cl. 435/91.2; 435/303.1

(57) ABSTRACT

Systems and methods for processing sample processing devices. The system can include a base plate adapted to rotate about a rotation axis. The base plate can include at least one first magnetic element. The system can further include an annular cover, and a sample processing device comprising at least one thermal process chamber. The annular cover can include an inner edge, an outer edge, and at least one second magnetic element. The method can include positioning the sample processing device between the base plate and the annular cover, such that the inner edge of the annular cover is positioned inwardly of the at least one thermal process chamber, and such that the at least one first magnetic element attracts the at least one second magnetic element to force the annular cover in a first direction along the z-axis, urging the sample processing device into contact with the base plate.

### FIELD

The present disclosure relates to systems and methods for using rotating sample processing devices to, e.g., amplify genetic materials, etc.

### BACKGROUND

Many different chemical, biochemical, and other reactions are sensitive to temperature variations. Examples of thermal processes in the area of genetic amplification include, but are not limited to, Polymerase Chain Reaction (PCR), Sanger sequencing, etc. One approach to reducing the time and cost of thermally processing multiple samples is to use a device including multiple chambers in which different portions of one sample or different samples can be processed simultaneously. Examples of some reactions that may require accurate chamber-to-chamber temperature control, comparable temperature transition rates, and/or rapid transitions between temperatures include, e.g., the manipulation of nucleic acid samples to assist in the deciphering of the genetic code. Nucleic acid manipulation techniques include amplification methods such as polymerase chain reaction (PCR); target polynucleotide amplification methods such as self-sustained sequence replication (3SR) and strand-displacement amplification (SDA); methods based on amplification of a signal attached to the target polynucleotide, such as “branched chain” DNA amplification; methods based on amplification of probe DNA, such as ligase chain reaction (LCR) and QB replicase amplification (QBR); transcription-based methods, such as ligation activated transcription (LAT) and nucleic acid sequence-based amplification (NASBA); and various other amplification methods, such as repair chain reaction (RCR) and cycling probe reaction (CPR). Other examples of nucleic acid manipulation techniques include, e.g., Sanger sequencing, ligand-binding assays, etc.

Some systems used to process rotating sample processing devices are described in U.S. Pat. No. 6,889,468 titled MODULAR SYSTEMS AND METHODS FOR USING SAMPLE PROCESSING DEVICES and U.S. Pat. No. 6,734,401 titled ENHANCED SAMPLE PROCESSING DEVICES SYSTEMS AND METHODS (Bedingham et al.).

### SUMMARY

Some embodiments of the present disclosure provide a system for processing sample processing devices. The system can include a base plate operatively coupled to a drive system, wherein the drive system rotates the base plate about a rotation axis, and wherein the rotation axis defines a z-axis. The system can further include a thermal structure operatively coupled to the base plate, wherein the thermal structure comprises a transfer surface exposed proximate a first surface of the base plate. The system can further include at least one first magnetic element operatively coupled to the base plate, and a sample processing device comprising at least one thermal process chamber. The system can further include an annular cover adapted to face the transfer surface. The annular cover can include a center, an inner edge, and an outer edge. The sample processing device can be adapted to be positioned between the base plate and the annular cover. The inner edge of the annular cover can be configured to be positioned inwardly of the at least one thermal process chamber, relative to the center of the annular cover, for example, when the sample processing device is positioned adjacent the annular cover. The system can further include at least one second magnetic element operatively coupled to the annular cover. The at least one second magnetic element can be configured to attract the at least one first magnetic element to force the annular cover in a first direction along the z-axis, such that at least a portion of the sample processing device is urged into contact with the transfer surface of the base plate.

Some embodiments of the present disclosure provide a system for processing sample processing devices. The system can include a base plate operatively coupled to a drive system, wherein the drive system rotates the base plate about a rotation axis, and wherein the rotation axis defines a z-axis. The system can further include a thermal structure operatively coupled to the base plate, wherein the thermal structure comprises a transfer surface exposed proximate a first surface of the base plate. The system can further include a first annulus of magnetic elements operatively coupled to the base plate, and a sample processing device comprising at least one thermal process chamber. The system can further include an annular cover adapted to face the transfer surface. The annular cover can include an inner edge and an outer edge. The inner edge can be positioned inwardly of the at least one thermal process chamber, and the sample processing device can be adapted to be positioned between the base plate and the annular cover. The system can further include a second annulus of magnetic elements operatively coupled to the annular cover. The second annulus of magnetic elements can be configured to attract the first annulus of magnetic elements to force the annular cover in a first direction along the z-axis, such that at least a portion of the sample processing device is urged into contact with the transfer surface of the base plate.

Some embodiments of the present disclosure provide a method for processing sample processing devices. The method can include providing a base plate operatively coupled to a drive system, and providing a thermal structure operatively coupled to the base plate. The thermal structure can include a transfer surface exposed proximate a first surface of the base plate. The method can further include providing a sample processing device comprising at least one thermal process chamber, and providing an annular cover facing the transfer surface. The annular cover can include an inner edge and an outer edge. The method can further include providing at least one first magnetic element operatively coupled to the base plate and at least one second magnetic element operatively coupled to the annular cover. The method can further include positioning the sample processing device between the base plate and the annular cover, such that the inner edge of the annular cover is positioned inwardly of the at least one thermal process chamber, and such that the at least one first magnetic element attracts the at least one second magnetic element to force the annular cover in a first direction along the z-axis, such that at least a portion of the sample processing device is urged into contact with the transfer surface of the base plate. The method can further include rotating the base plate about a rotation axis, wherein the rotation axis defines a z-axis.

Other features and aspects of the present disclosure will become apparent by consideration of the detailed description and accompanying drawings.

### BRIEF DESCRIPTION OF THE DRAWINGS

FIG. 1 is an exploded perspective view of a system according to one embodiment of the present disclosure, the system including a cover, a sample processing device, and a base plate.

FIG. 2 is an assembled perspective cross-sectional view of the system of FIG. 1.

FIG. 3 is an assembled close-up cross-sectional view of the system of FIGS. 1-2.

FIG. 4 is a bottom plan view of the cover of FIGS. 1-3.

FIG. 5 is a cross-sectional view of a portion of the sample processing device of FIGS. 1-3, taken along line 5-5 of FIG. 1.

FIG. 6 is close-up plan view of a portion of the sample processing device of FIGS. 1-3 and 5.

FIG. 7 is an exploded perspective view of a system according to another embodiment of the present disclosure, the system including a cover, a sample processing device, and a base plate.

FIG. 8 is an assembled close-up cross-sectional view of the system of FIG. 7.

FIG. 9 is an exploded perspective view of a system according to another embodiment of the present disclosure, the system including a cover, a sample processing device, and a base plate.

FIG. 10 is an assembled close-up cross-sectional view of the system of FIG. 9.

FIG. 11 is a perspective cross-sectional view of a portion of the base plate of FIG. 1, taken along line 11-11 in FIG. 1, showing one embodiment of a resiliently biased thermal structure.

FIG. 12 is a perspective view of one exemplary biasing member that may be used in connection with the systems of the present disclosure.

FIG. 13 is a close-up cross-sectional view of a system according to another embodiment of the present disclosure, the system including a cover, a sample processing device, and a base plate, the base plate including a thermal structure having a shaped transfer according to one embodiment of the present disclosure.

FIG. 14 is a diagram depicting the radial cross-sectional profile of a shaped thermal transfer surface according to another embodiment of the present disclosure.

FIG. 15 is a diagram depicting the radial cross-sectional profile of a shaped thermal transfer surface according to another embodiment of the present disclosure.

FIGS. 16A-16C depict alternative edge structures for compression rings on a cover according to other embodiments of the present disclosure.

### DETAILED DESCRIPTION

Before any embodiments of the present disclosure are explained in detail, it is to be understood that the invention is not limited in its application to the details of construction and the arrangement of components set forth in the following description or illustrated in the following drawings. The invention is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Unless specified or limited otherwise, the terms “mounted,” and “coupled” and variations thereof are used broadly and encompass both direct and indirect mountings and couplings. Further, “coupled” is not restricted to physical or mechanical couplings. It is to be understood that other embodiments may be utilized, and structural or logical changes may be made without departing from the scope of the present disclosure. Furthermore, terms such as “front,” “rear,” “top,” “bottom,” and the like are only used to describe elements as they relate to one another, but are in no way meant to recite specific orientations of the apparatus, to indicate or imply necessary or required orientations of the apparatus, or to specify how the invention described herein will be used, mounted, displayed, or positioned in use.

The present disclosure generally relates to annular compression systems and methods for sample processing devices. Such annular compression systems can include an open area (e.g., an open central area), such that the annular compression system can perform and/or facilitate the desired thermal control and rotation functions for the sample processing device, while allowing access to at least a portion of the sample processing device. For example, some existing systems cover a top surface of a sample processing device in order to hold the sample processing device onto a rotating base plate and/or to thermally control and isolate portions of the sample processing device (e.g., from one another and/or ambience). The annular compression systems and methods of the present disclosure, however, provide the desired positioning and holding functions as well as the desired thermal control functions, while also allowing a portion of the sample processing device to be exposed to other devices or systems for which it may be desirable to have direct access to the sample processing device. For example, in some embodiments, sample delivery (e.g., manual or automatic pipetting) can be accomplished after the sample processing device has already been positioned between an annular cover and a base plate. By way of further example, in some embodiments, a portion of the sample processing device can be optically accessible (e.g., to electromagnetic radiation), for example, which can enable more efficient laser addressing of the sample processing device, or which can be used for optical interrogation (e.g., absorption, reflectance, fluorescence, etc.). Such laser addressing can be used, for example, for fluid (e.g., microfluidic) manipulation of a sample in the sample processing device.

Furthermore, in some embodiments, the annular compression systems and methods of the present disclosure can enable unique temperature control of various portions of the sample processing device. For example, fluid (e.g., air) can be moved over an exposed surface of the sample processing device in areas that are desired to be rapidly cooled, while the areas that are desired to be heated or maintained at a desired temperature can be covered and isolated from other portions of the sample processing device and/or from ambience.

In addition, in some embodiments, annular compression systems and methods of the present disclosure can allow a portion of the sample processing device to be exposed to interact with other (e.g., external or internal) devices or equipment, such as robotic workstations, pipettes, interrogation instruments, and the like, or combinations thereof. Similarly, the annular compression systems and methods of the present disclosure can protect desired portions of the sample processing device from contact.

As a result, “accessing” at least a portion of a sample processing device can refer to a variety of processing steps and can include, but is not limited to, physically or mechanically accessing the sample processing device (e.g., delivering or retrieving a sample via direct or indirect contact, moving or manipulating a sample in the sample processing device via direct or indirect contact, etc.); optically accessing the sample processing device (e.g., laser addressing); thermally accessing the sample processing device (e.g., selectively heating or cooling an exposed portion of the sample processing device); and the like; and combinations thereof.

The present disclosure provides methods and systems for sample processing devices that can be used in methods that involve thermal processing, e.g., sensitive chemical processes such as polymerase chain reaction (PCR) amplification, transcription-mediated amplification (TMA), nucleic acid sequence-based amplification (NASBA), ligase chain reaction (LCR), self-sustaining sequence replication, enzyme kinetic studies, homogeneous ligand binding assays, and more complex biochemical or other processes that require precise thermal control and/or rapid thermal variations. The sample processing systems are capable of providing simultaneous rotation of the sample processing device in addition to effecting control over the temperature of sample materials in process chambers on the devices.

Some examples of suitable sample processing devices that may be used in connection with the methods and systems of the present disclosure may be described in, e.g., commonly-assigned U.S. Patent Publication No. 2007/0010007 titled SAMPLE PROCESSING DEVICE COMPRESSION SYSTEMS AND METHODS (Aysta et al.); U.S. Patent Publication No. 2007/0009391 titled COMPLIANT MICROFLUIDIC SAMPLE PROCESSING DISKS (Bedingham et al.); U.S. Patent Publication No. 2008/0050276 titled MODULAR SAMPLE PROCESSING APPARATUS KITS AND MODULES (Bedingham et al.); U.S. Pat. No. 6,734,401 titled ENHANCED SAMPLE PROCESSING DEVICES SYSTEMS AND METHODS (Bedingham et al.) and U.S. Pat. No. 7,026,168 titled SAMPLE PROCESSING DEVICES (Bedingham et al.). Other useable device constructions may be found in, e.g., U.S. Pat. No. 7,435,933 (Bedingham et al.) titled ENHANCED SAMPLE PROCESSING DEVICES, SYSTEMS AND METHODS; U.S. Provisional Patent Application Ser. No. 60/237,151 filed on Oct. 2, 2000 and entitled SAMPLE PROCESSING DEVICES, SYSTEMS AND METHODS (Bedingham et al.); and U.S. Pat. No. 6,814,935 titled SAMPLE PROCESSING DEVICES AND CARRIERS (Harms et al.). Other potential device constructions may be found in, e.g., U.S. Pat. No. 6,627,159 titled CENTRIFUGAL FILLING OF SAMPLE PROCESSING DEVICES (Bedingham et al.); PCT Patent Publication No. WO 2008/134470 titled METHODS FOR NUCLEIC ACID AMPLIFICATION (Parthasarathy et al.); and U.S. Patent Publication No. 2008/0152546 titled ENHANCED SAMPLE PROCESSING DEVICES, SYSTEMS AND METHODS (Bedingham et al.).

Some embodiments of the sample processing systems of the present disclosure can include base plates attached to a drive system in a manner that provides for rotation of the base plate about an axis of rotation. When a sample processing device is secured to the base plate, the sample processing device can be rotated with the base plate. The base plate can include at least one thermal structure that can be used to heat portions of the sample processing device and may include a variety of other components as well, e.g., temperature sensors, resistance heaters, thermoelectric modules, light sources, light detectors, transmitters, receivers, etc.

Other elements and features of systems and methods for processing sample processing devices can be found in patent application Ser. No. ______ (Attorney Docket No. 65917US002), filed on even date herewith, which is incorporated herein by reference in its entirety.

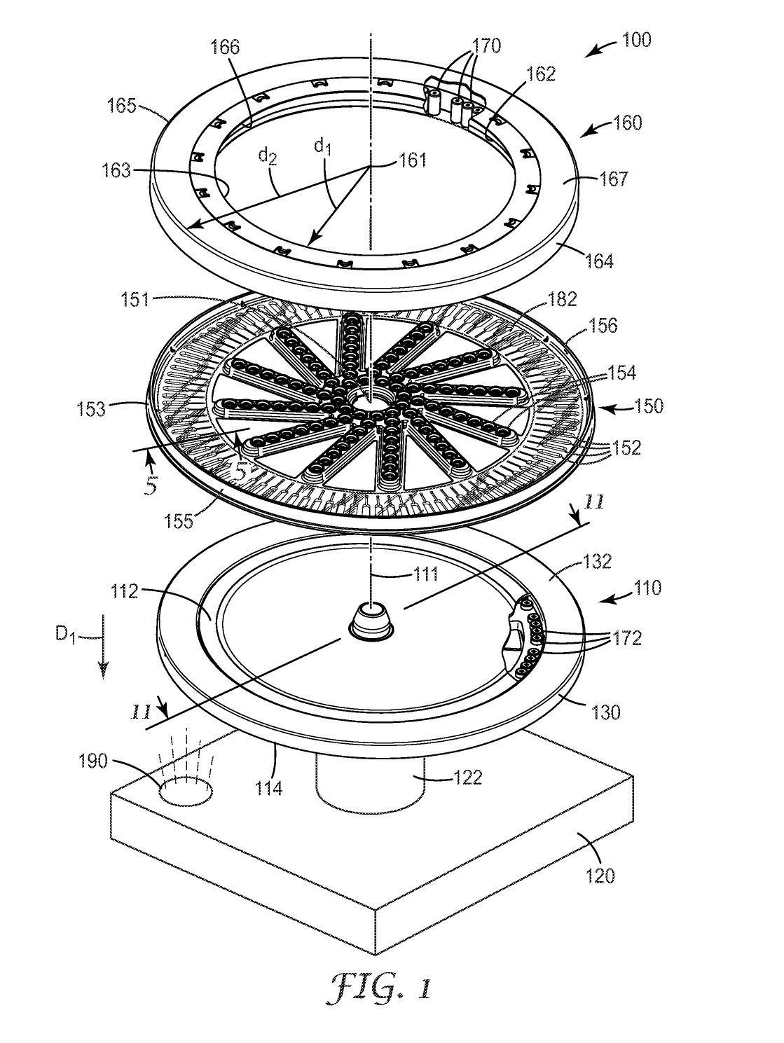

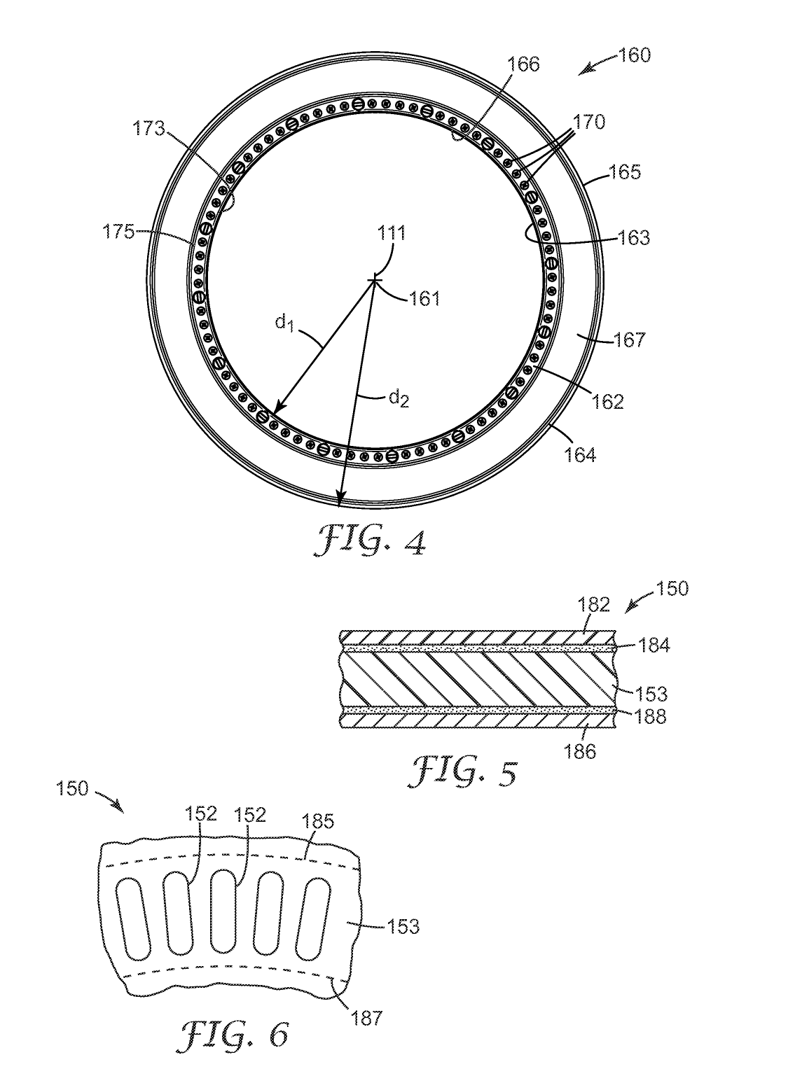

One illustrative sample processing system 100 is shown in FIGS. 1-6 and 11-12. As shown in FIGS. 1-3, the system 100 can include a base plate 110 that rotates about an axis of rotation 111. The base plate 110 can also be attached to a drive system 120, for example, via a shaft 122. It will, however, be understood that the base plate 110 may be coupled to the drive system 120 through any suitable alternative arrangement, e.g., belts or a drive wheel operating directly on the base plate 110, etc.

Also depicted in FIG. 1 is a sample processing device 150 and an annular cover 160 that can be used in connection with the base plate 110, as will be described herein. Systems of the present disclosure may not actually include a sample processing device as, in some instances, sample processing devices are consumable devices that are used to perform a variety of tests, etc. and then discarded. As a result, the systems of the present disclosure may be used with a variety of different sample processing devices.

As shown in FIGS. 1-3, the depicted base plate 110 includes a thermal structure 130 that can include a thermal transfer surface 132 exposed on the top surface 112 of the base plate 110. By “exposed” it is meant that the transfer surface 132 of the thermal structure 130 can be placed in physical contact with a portion of a sample processing device 150 such that the thermal structure 130 and the sample processing device 150 are thermally coupled to transfer thermal energy via conduction. In some embodiments, the transfer surface 132 of the thermal structure 130 can be located directly beneath selected portions of a sample processing device 150 during sample processing. For example, in some embodiments, the selected portions of the sample processing device 150 can include one or more process chambers, such as thermal process chambers 152. The process chambers can include those discussed in, e.g., U.S. Pat. No. 6,734,401 titled ENHANCED SAMPLE PROCESSING DEVICES SYSTEMS AND METHODS (Bedingham et al.). By way of further example, the sample processing device 150 can include various features and elements, such as those described in U.S. Patent Publication No. 2007/0009391 titled COMPLIANT MICROFLUIDIC SAMPLE PROCESSING DISKS (Bedingham et al.).

As a result, by way of example only, the sample processing device 150 illustrated in FIGS. 1-3 and 5-6 can include one or more input wells and/or other chambers (sometimes referred to as “non-thermal” chambers or “non-thermal” process chambers) 154 positioned in fluid communication with the thermal process chambers 152. For example, in some embodiments, a sample can be loaded onto the sample processing device 150 via the input wells 154 and can then be moved via channels (e.g., microfluidic channels) and/or valves to other chambers and/or ultimately to the thermal process chambers 152.

In some embodiments, as shown in FIGS. 1-3, the input wells 154 can be positioned between a center 151 of the sample processing device 150 and at least one of the thermal process chambers 152. In addition, the annular cover 160 can be configured to allow access to a portion of the sample processing device 150 that includes the input well(s) 154, such that the input well(s) 154 can be accessed when the cover 160 is positioned adjacent to or coupled to the sample processing device 150.

As shown in FIGS. 1-4, the annular cover 160 can, together with the base plate 110, compress a sample processing device 150 located therebetween, for example, to enhance thermal coupling between the thermal structure 130 on the base plate 110 and the sample processing device 150. In addition, the annular cover 160 can function to hold and/or maintain the sample processing device 150 on the base plate 110, such that the sample processing device 150 and/or the cover 160 can rotate with the base plate 110 as it is rotated about axis 111 by drive system 120. The rotation axis 111 can define a z-axis of the system 100.

As used herein, the term “annular” or derivations thereof can refer to a structure having an outer edge and an inner edge, such that the inner edge defines an opening. For example, an annular cover can have a circular or round shape (e.g., a circular ring) or any other suitable shape, including, but not limited to, triangular, rectangular, square, trapezoidal, polygonal, etc., or combinations thereof. Furthermore, an “annulus” of the present invention need not necessarily be symmetrical, but rather can be an asymmetrical or irregular shape; however, certain advantages may be possible with symmetrical and/or circular shapes.

The compressive forces developed between the base plate 110 and the cover 160 may be accomplished using a variety of different structures or combination of structures. One exemplary compression structure depicted in the embodiment of FIGS. 1-6 are magnetic elements 170 located on (or at least operatively coupled to) the cover 160 and corresponding magnetic elements 172 located on (or at least operatively coupled to) the base plate 110. Magnetic attraction between the magnetic elements 170 and 172 may be used to draw the cover 160 and the base plate 110 towards each other, thereby compressing, holding, and/or deforming a sample processing device 150 located therebetween. As a result, the magnetic elements 170 and 172 can be configured to attract each other to force the annular cover 160 in a first direction D₁ (see FIG. 1) along the z-axis of the system 100, such that at least a portion of the sample processing device 150 is urged into contact with the transfer surface 132 of the base plate 110.

As used herein, a “magnetic element” is a structure or article that exhibits or is influenced by magnetic fields. In some embodiments, the magnetic fields can be of sufficient strength to develop the desired compressive force that results in thermal coupling between a sample processing device 150 and the thermal structure 130 of the base plate 110 as discussed herein. The magnetic elements can include magnetic materials, i.e., materials that either exhibit a permanent magnetic field, materials that are capable of exhibiting a temporary magnetic field, and/or materials that are influenced by permanent or temporary magnetic fields.

Some examples of potentially suitable magnetic materials include, e.g., magnetic ferrite or “ferrite” which is a substance including mixed oxides of iron and one or more other metals, e.g., nanocrystalline cobalt ferrite. However, other ferrite materials may be used. Other magnetic materials which may be used in the system 100 may include, but are not limited to, ceramic and flexible magnetic materials made from strontium ferrous oxide which may be combined with a polymeric substance (such as, e.g., plastic, rubber, etc.); NdFeB (this magnetic material may also include Dysprosium); neodymium boride; SmCo (samarium cobalt); and combinations of aluminum, nickel, cobalt, copper, iron, titanium, etc.; as well as other materials. Magnetic materials may also include, for example, stainless steel, paramagnetic materials, or other magnetizable materials that may be rendered sufficiently magnetic by subjecting the magnetizable material to a sufficient electric and/or magnetic field.

In some embodiments, the magnetic elements 170 and/or the magnetic elements 172 can include strongly ferromagnetic material to reduce magnetization loss with time, such that the magnetic elements 170 and 172 can be coupled with a reliable magnetic force, without substantial loss of that force over time.

Furthermore, in some embodiments, the magnetic elements of the present disclosure may include electromagnets, in which the magnetic fields can be switched on and off between a first magnetic state and a second non-magnetic state to activate magnetic fields in various areas of the system 100 in desired configurations when desired.

In some embodiments, the magnetic elements 170 and 172 can be discrete articles operatively coupled to the cover 160 and the base plate 110, as depicted in the embodiment of FIGS. 1-6 and 11-12 (in which the magnetic elements 170 and 172 are individual cylindrically-shaped articles). However, in some embodiments, the base plate 110, the thermal structure 130, and/or the cover 160 can include sufficient magnetic material (e.g., molded or otherwise provided in the structure of the component), such that separate discrete magnetic elements are not required. In some embodiments, a combination of discrete magnetic elements and sufficient magnetic material (e.g., molded or otherwise) can be employed.

As shown in FIGS. 1-4, the annular cover 160 includes a center 161, which, in the embodiment illustrated in FIGS. 1-6 and 11-12 is in line with the rotation axis 111 when the cover 160 is coupled to the base plate 110, an inner edge 163 that at least partially defines an opening 166, and an outer edge 165. As described above, the opening 166 can facilitate accessing at least a portion of the sample processing device 150 (e.g., a portion comprising the input wells 154), for example, even when the annular cover 160 is positioned adjacent to or coupled to the sample processing device 150. As shown in FIGS. 1-3, the inner edge 163 of the annular cover 160 can be configured to be positioned inwardly (e.g., radially inwardly) of the thermal process chambers 152, relative to the center 161 of the annular cover 160, for example, when the annular cover 160 is positioned adjacent the sample processing device 150. In addition, the inner edge 163 of the annular cover 160 can be configured to be positioned radially outwardly of the input wells 154. Furthermore, in some embodiments, as shown in FIGS. 1-4, the outer edge 165 of the annular cover 160 can be configured to be positioned outwardly (e.g., radially outwardly) of the thermal process chambers 152 (and also outwardly of the input wells 154).

The inner edge 163 can be positioned a first distance d₁ (e.g., a first radial distance or “first radius”) from the center 161 of the annular cover 160. In such embodiments, if the annular cover 160 has a substantially circular ring shape, the opening 166 can have a diameter equal to twice the first distance d₁. In addition, the outer edge 165 can be positioned a second distance d₂ (e.g., a second radial distance or “second radius”) from the center 161 of the annular cover 160. In some embodiments, the first distance d₁ can be at least about 50% of the second distance. In some embodiments, at least about 60%, and in some embodiments, at least about 70%. In addition, in some embodiments, the first distance d₁ can be no greater than about 95% of the second distance, in some embodiments, no greater than about 85%, and in some embodiments, no greater than about 80%. In some embodiments, the first distance d₁ can be about 75% of the second distance d₂.

Furthermore, in some embodiments, the outer edge 165 can be positioned a distance d₂ (e.g., a radial distance) from the center 161, which can define a first area, and in some embodiments, the area of the opening 166 can be at least about 30% of the first area, in some embodiments, at least about 40%, and in some embodiments, at least about 50%. In some embodiments, the opening 166 can be no greater than about 95% of the first area, in some embodiments, no greater than about 75%, and in some embodiments, no greater than about 60%. In some embodiments, the opening 166 can be about 53% of the first area.

In addition, the annular cover 160 can include an inner wall 162 (e.g., an “inner circumferential wall” or “inner radial wall”; which can function as an inner compression ring, in some embodiments, as described below) and an outer wall 164 (e.g., an “outer circumferential wall” or “outer radial wall”; which can function as an outer compression ring, in some embodiments, as described below). In some embodiments, inner and outer walls 162 and 164 can include or define the inner and outer edges 163 and 165, respectively, such that the inner wall 162 can be positioned inwardly (e.g., radially inwardly) of the thermal process chambers 152, and the outer wall 164 can be positioned outwardly (e.g., radially outwardly) of the thermal process chambers 152. As further shown in FIGS. 1-4, in some embodiments, the inner wall 162 can include the magnetic elements 170, such that the magnetic elements 170 form a portion of or are coupled to the inner wall 162. For example, in some embodiments, the magnetic elements 170 can be embedded (e.g., molded) in the inner wall 162. As shown in FIGS. 1-4, the annular cover 160 can further include an upper wall 167 that can be positioned to cover a portion of the sample processing device 150, such as a portion that comprises the thermal process chambers 152.

As shown in FIGS. 1 and 2, in some embodiments, the upper wall 167 can extend inwardly (e.g., radially inwardly) of the inner wall 162 and the magnetic elements 170. In the embodiment illustrated in FIGS. 1-4, the upper wall 167 does not extend much inwardly of the inner wall 162. However, in some embodiments, the upper wall 167 can extend further inwardly of the inner wall 162 and/or the magnetic elements 170 (e.g., toward the center 161 of the cover 160), for example, such that the size of the opening 166 is smaller than what is depicted in FIGS. 1-4. Furthermore, in some embodiments, the upper wall 167 can define the inner edge 163 and/or the outer edge 165.

In some embodiments, at least a portion of the cover 160, such as one or more of the inner wall 162, the outer wall 164, and the upper wall 167, can be optically clear. As used herein, the phrase “optically clear” can refer to an object that is transparent to electromagnetic radiation ranging from the infrared to the ultraviolet spectrum (e.g., from about 10 nm to about 10 μm (10,000 nm)); however, in some embodiments, the phrase “optically clear” can refer to an object that is transparent to electromagnetic radiation in the visible spectrum (e.g., about 400 nm to about 700 nm). In some embodiments, the phrase “optically clear” can refer to an object with a transmittance of at least about 80% within the wavelength ranges above.

Such configurations of the annular cover 160 can function to effectively or substantially isolate the thermal process chambers 152 of the sample processing device 150 when the cover 160 is coupled to or positioned adjacent the sample processing device 150. For example, the cover 160 can physically, optically, and/or thermally isolate a portion of the sample processing device 150, such as a portion comprising the thermal process chambers 152. In some embodiments, as shown in FIGS. 1 and 6, the sample processing device 150 can include one or more thermal process chambers 152, and further, in some embodiments, the one or more thermal process chambers 152 can be arranged in an annulus about the center 151 of the sample processing device 150, which can sometimes be referred to as an “annular processing ring.” In such embodiments, the annular cover 160 can be adapted to cover and/or isolate a portion of the sample processing device 150 that includes the annular processing ring or the thermal process chambers 152. For example, the annular cover 160 includes the inner wall 162, the outer wall 164, and the upper wall 167 to cover and/or isolate the portion of the sample processing device 150 that includes the thermal process chambers 152. In some embodiments, one or more of the inner wall 162, the outer wall 164, and the upper wall 167 can be a continuous wall, as shown, or can be formed of a plurality of portions that together function as an inner or outer wall (or inner or outer compression ring), or an upper wall. In some embodiments, enhanced physical and/or thermal isolation can be obtained when at least one of the inner wall 162, the outer wall 164 and the upper wall 167 is a continuous wall.

In addition, in some embodiments, the ability of the annular cover 160 to cover and effectively thermally isolate the thermal process chambers 152 from ambience and/or from other portions of the system 100 can be important, because otherwise, as the base plate 110 and the sample processing device 150 are rotated about the rotation axis 111, air can be caused to move quickly past the thermal process chambers 152, which, for example, can undesirably cool the thermal process chambers 152 when it is desired for the chambers 152 to be heated. Thus, in some embodiments, depending on the configuration of the sample processing device 150, one or more of the inner wall 162, the upper wall 167 and the outer wall 164 can be important for thermal isolation.

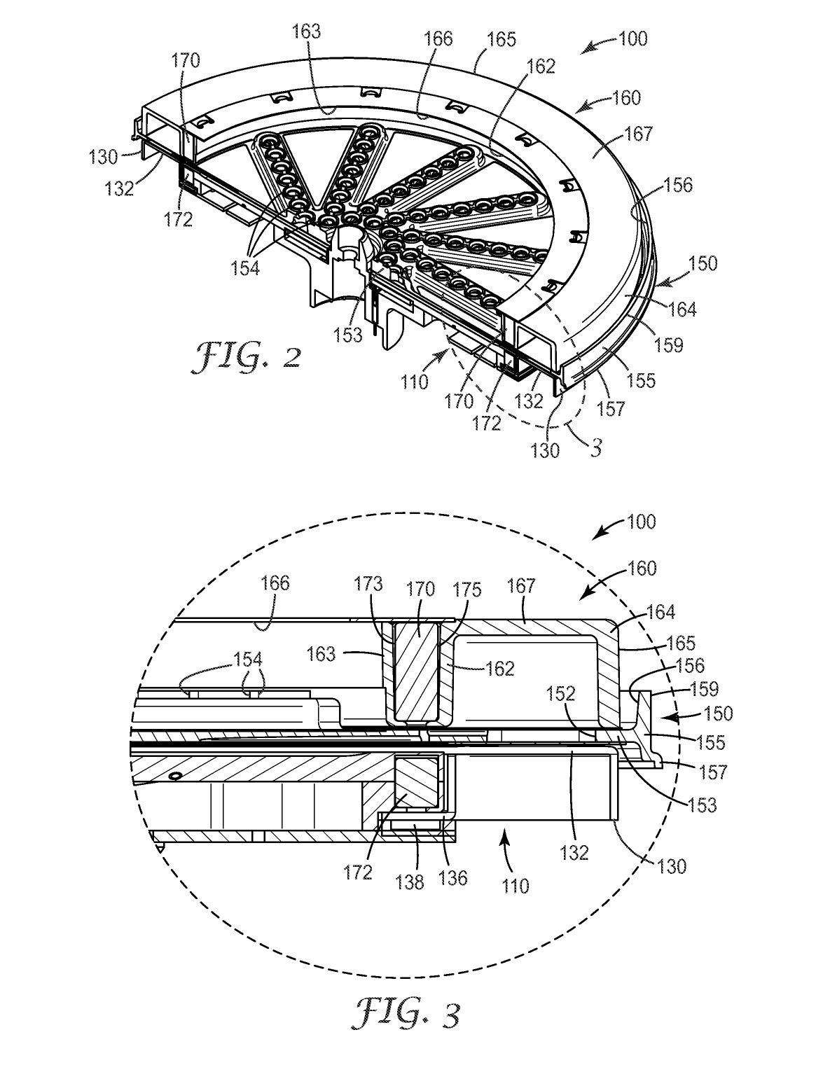

As shown in FIGS. 1-3 and 5-6, in some embodiments, the sample processing device 150 can also include a device housing or body 153, and in some embodiments, the body 153 can define the input wells 154 or other chambers, any channels, the thermal process chambers 152, etc. In addition, in some embodiments, the body 153 of the sample processing device 150 can include an outer lip, flange or wall 155. In some embodiments, as shown in FIGS. 1-3, the outer wall 155 can include a portion 157 adapted to cooperate with the base plate 110 and a portion 159 adapted to cooperate with the annular cover 160. For example, as shown in FIGS. 2 and 3, the annular cover 160 (e.g., the outer wall 164) can be dimensioned to be received within the area circumscribed by the outer wall 155 of the sample processing device 150. As a result, in some embodiments, the outer wall 155 of the sample processing device 150 can cooperate with the annular cover 160 to cover and/or isolate the thermal process chambers 152. Such cooperation can also facilitate positioning of the annular cover 160 with respect to the sample processing device 150 such that the thermal process chambers 152 are protected and covered without the annular cover 160 pressing down on or contacting any of the thermal process chambers 152.

In some embodiments, the outer wall 155 of the sample processing device 150 and the one or more input wells 154 formed in the body 153 of the sample processing device 150 can effectively define a recess (e.g., an annular recess) 156 in the sample processing device 150 (e.g., in a top surface of the sample processing device 150) in which at least a portion of the annular cover 160 can be positioned. For example, as shown in FIGS. 1-3, the inner wall 162 (e.g., including the magnetic elements 170) and the outer wall 164 can be positioned in the recess 156 of the sample processing device 150 when the annular cover 160 is positioned over or coupled to the sample processing device 150. As a result, in some embodiments, the outer wall 155, the input wells 154 and/or the recess 156 can provide reliable positioning of the cover 160 with respect to the sample processing device 150.

In some embodiments, as shown in FIGS. 1-4, the magnetic elements 170 can be arranged in an annulus, and the annulus or portion of the cover 160 that includes the magnetic elements 170 can include an inner edge (e.g., an inner radial edge) 173 and an outer edge (e.g., an outer radial edge) 175. As shown in FIGS. 1-3, the cover 160 and/or the magnetic elements 170 can be configured, such that both the inner edge 173 and the outer edge 175 can be positioned inwardly (e.g., radially inwardly) with respect to the thermal process chambers 152.

As a result, in some embodiments, the magnetic elements 170 can be restricted to an area of the cover 160 where the magnetic elements 170 are positioned outwardly (e.g., radially outwardly) of the input wells 154 (or other protrusions, chambers, recesses, or formations in the body 153) and inwardly (e.g., radially inwardly) of the thermal process chambers 152. In such configurations, the magnetic elements 170 can be said to be configured to maximize the open area of the sample processing device 150 that is available for access by other devices or for other functions. In addition, in such embodiments, the magnetic elements 170 can be positioned so as not to interrupt or disturb the processing of a sample positioned in the thermal process chambers 152.

In some embodiments, as shown in FIGS. 1-4, the magnetic elements 170 of the cover 160 can form at least a portion of or be coupled to the inner wall 162, such that the magnetic elements 170 can function as at least a portion of the inner compression ring 162 to compress, hold, and/or deform the sample processing device 150 against the thermal transfer surface 132 of the thermal structure 130 of the base plate 110. As shown in FIGS. 1-4, one or both of the magnetic elements 170 and 172 can be arranged in an annulus, for example, about the rotation axis 111. Furthermore, in some embodiments, at least one of the magnetic elements 170 and 172 can include a substantially uniform distribution of magnetic force about such an annulus.

In addition, the arrangement of the magnetic elements 170 in the cover 160 and the corresponding arrangement of the magnetic elements 172 in the base plate 110 can provide additional positioning assistance for the cover 160 with respect to one or both of the sample processing device 150 and the base plate 110. For example, in some embodiments, the magnetic elements 170 and 172 can each include sections of alternating polarity and/or a specific configuration or arrangement of magnetic elements, such that the magnetic elements 170 of the cover 160 and the magnetic elements 172 of the base plate 110 can be “keyed” with respect to each other to allow the cover 160 to reliably be positioned in a desired orientation (e.g., angular position relative to the rotation axis 111) with respect to at least one of the sample processing device 150 and the base plate 110.

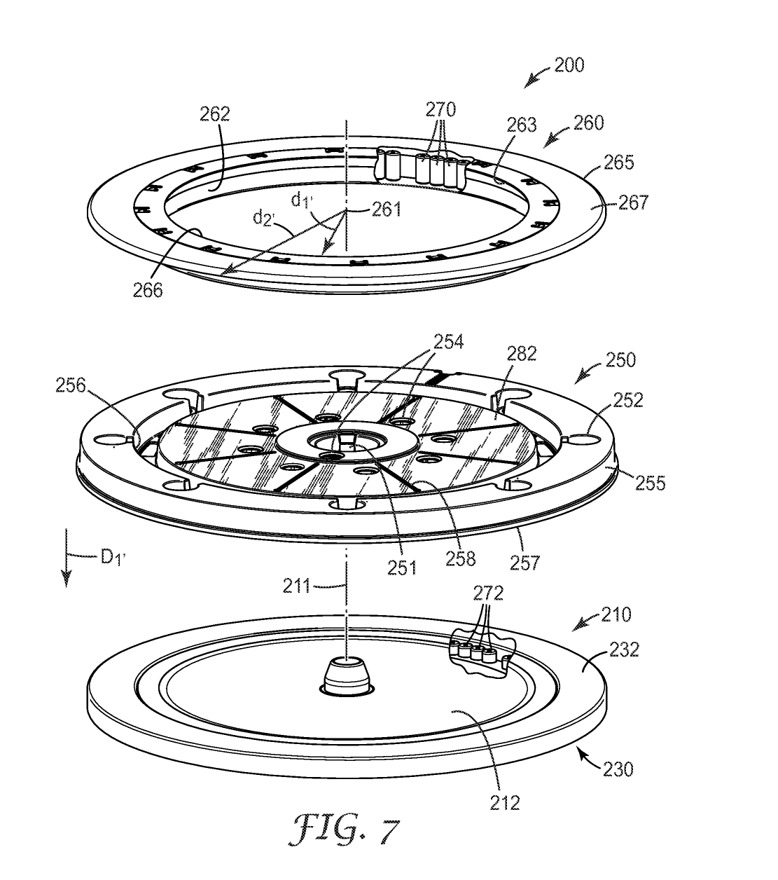

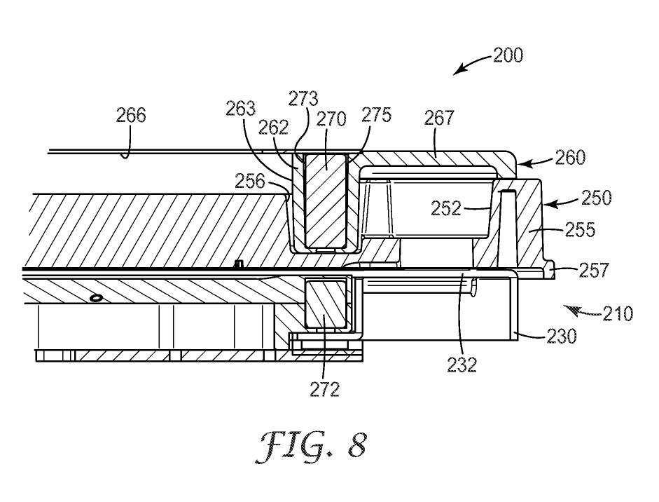

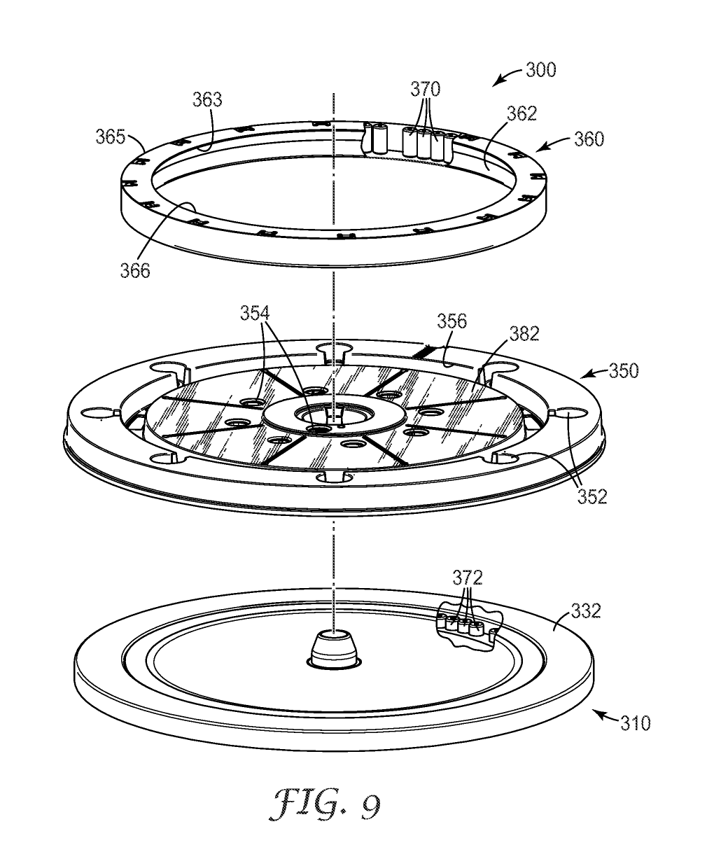

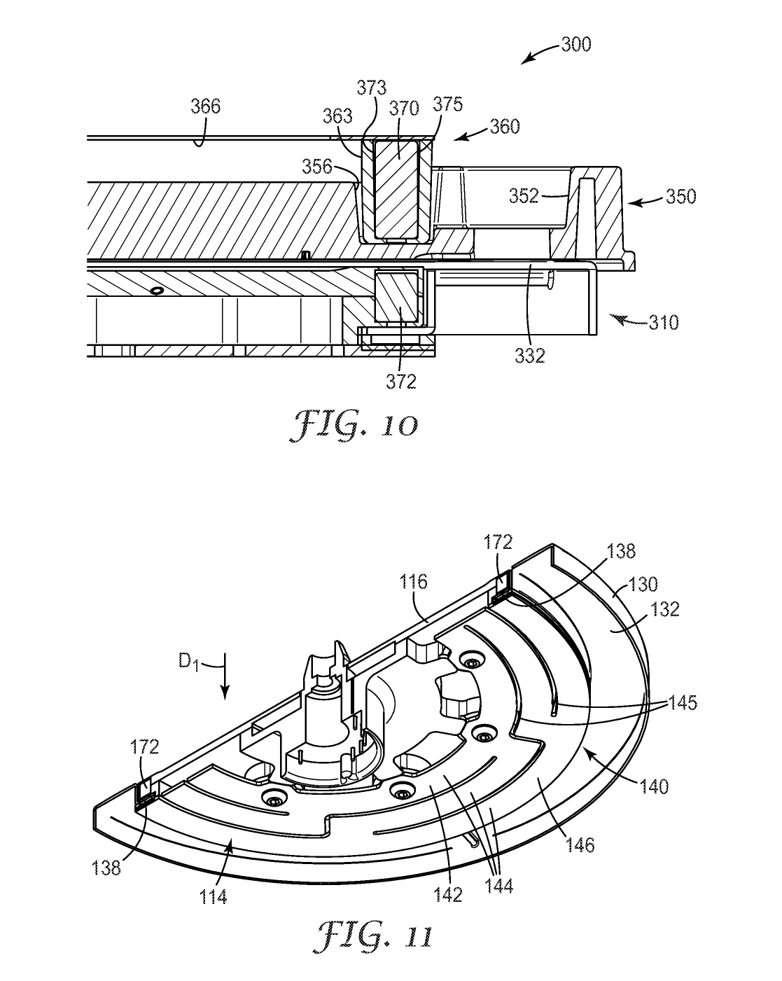

In some embodiments, as described below and illustrated in FIGS. 7-8, the annular cover 160 may not include an outer wall 164. In such embodiments, the thermal process chambers 152 may be exposed and accessible, or the upper wall 167 alone may cover that portion of the sample processing device 150. Furthermore, as described below and illustrated in FIGS. 9-10, in some embodiments, the annular cover 160 may not include an upper wall 167. In some embodiments, thermal isolation of the thermal process chambers 152, if desired, can largely be provided by the sample processing device 150 alone. As will be described below with respect to FIGS. 7-10, the annular covers of the present disclosure can be adapted to cooperate with a variety of sample processing devices. As a result, certain annular covers may be more useful in combination with some sample processing devices than others.

In some embodiments, compliance of sample processing devices of the present disclosure may be enhanced if the devices include annular processing rings that are formed as composite structures including cores and covers attached thereto using pressure sensitive adhesives. The sample processing device 150 shown in FIGS. 1-6 is an example of one such composite structure. As shown in FIGS. 1 and 5, in some embodiments, the sample processing device 150 can include the body 153 to which covers 182 and 186 are attached using adhesives (e.g., pressure sensitive adhesives) 184 and 188 (respectively). Where process chambers (e.g., thermal process chambers 152) are provided in a circular array (as depicted in FIGS. 1 and 6) that is formed by a composite structure such as that seen in FIG. 5, the thermal process chambers 152 and covers 182 and 186 can at least partially define a compliant annular processing ring that is adapted to conform to the shape of the underlying thermal transfer surface 132 when the sample processing device 150 is forced against the transfer surface 132, such as a shaped thermal transfer surface 132. In such embodiments, the compliance can be achieved with some deformation of the annular processing ring while maintaining the fluidic integrity of the thermal process chambers or any other fluidic passages or chambers in the sample processing device 150 (i.e., without causing leaks).

The body 153 and the different covers 182 and 186 used to seal any fluid structures (such as the thermal process chambers 152) in the sample processing device 150 may be manufactured of any suitable material or materials. Examples of suitable materials may include, e.g., polymeric materials (e.g., polypropylene, polyester, polycarbonate, polyethylene, etc.), metals (e.g., metal foils), etc. The covers can, but not necessarily, be provided in generally flat sheet-like pieces of, e.g., metal foil, polymeric material, multi-layer composite, etc. In some embodiments, the materials selected for the body 153 and the cover(s) 182 and/or 186 can exhibit good water barrier properties.

In some embodiments, at least one of the covers 182 and 186 can be constructed of a material or materials that substantially transmit electromagnetic energy of selected wavelengths. For example, in some embodiments, one or both of the covers 182 and 186 can be optically clear. By way of further example, in some embodiments, one or both of the covers 182 and 186 can be constructed of a material that allows for visual or machine monitoring of fluorescence or color changes within the thermal process chambers 152.

In some embodiments, at least one of the covers 182 and 186 can include a metallic layer, e.g., a metallic foil. If provided as a metallic foil, the cover 182 or 186 can include a passivation layer on the surface that faces the interior of the fluid structures to prevent contact between the sample materials and the metal. Such a passivation layer may also function as a bonding structure where it can be used in, e.g., hot melt bonding of polymers. As an alternative to a separate passivation layer, any adhesive layer used to attach the cover to the body 153 may also serve as a passivation layer to prevent contact between the sample materials and any metals in the cover.

In some embodiments, one cover 182 or 186 can be manufactured of a polymeric film (e.g., polypropylene) while the other cover 186 or 182 on the opposite side of the device 150 can include a metallic layer (e.g., a metallic foil layer of aluminum, etc.). For example, in such an embodiment, the cover 182 can transmit electromagnetic radiation of selected wavelengths, e.g., the visible spectrum, the ultraviolet spectrum, etc. into and/or out of the process chambers (e.g., thermal process chambers 152) while the metallic layer of cover 186 can facilitate thermal energy transfer into and/or out of the process chambers using thermal structures/surfaces as described herein.

The covers 182 and 186 can be coupled to the body 153 by any suitable technique or techniques, e.g., melt bonding, adhesives, combinations of melt bonding and adhesives, etc. If melt bonded, the cover and the surface to which it is attached can include, e.g., polypropylene or some other melt bondable material, to facilitate melt bonding. In some embodiments, the covers 182 and 186 can be coupled using pressure sensitive adhesive. The pressure sensitive adhesive may be provided in the form of a layer of pressure sensitive adhesive that, in some embodiments, can be provided as a continuous, unbroken layer between the cover and the opposing surface of the body 153. Examples of some potentially suitable attachment techniques, adhesives, etc. may be described in, e.g., U.S. Pat. No. 6,734,401 titled ENHANCED SAMPLE PROCESSING DEVICES SYSTEMS AND METHODS (Bedingham et al.) and U.S. Pat. No. 7,026,168 titled SAMPLE PROCESSING DEVICES (Bedingham et al.).

Pressure sensitive adhesives can exhibit viscoelastic properties that in some embodiments can allow for some movement of one or more of the covers 182 and/or 186 relative to the underlying body 153 to which the covers 182 and/or 186 are attached. The movement may be the result of deformation of the annular processing ring to, e.g., conform to a shaped transfer surface, such as those described in greater detail below. The relative movement may also be the result of different thermal expansion rates between the covers 182, 186 and the body 153. Regardless of the cause of the relative movement between covers and bodies in the sample processing devices of the present disclosure, in some embodiments, the viscoelastic properties of the pressure sensitive adhesive can allow the process chambers (e.g., the thermal process chambers 152) and other fluid features of the fluid structures to retain their fluidic integrity (i.e., they do not leak) in spite of the deformation.

Sample processing devices that include annular processing rings formed as composite structures using covers attached to bodies with viscoelastic pressure sensitive adhesives may, as described herein, exhibit compliance in response to forces applied to conform the annular processing rings to shaped transfer surfaces. Compliance of annular processing rings in sample processing devices used in connection with the present disclosure may alternatively be provided by, e.g., locating the process chambers in an (e.g., circular) array within the annular processing ring in which a majority of the area is occupied by voids in the body 153. For example, as shown in FIG. 1, the thermal process chambers 152 themselves may be formed by voids in the body 153 that are closed by one or more of the covers 182 and 186 attached to the body 153.

FIG. 6 is a close-up plan view of a portion of one major surface of the sample processing device 150 of the present disclosure. The portion of the device 150 depicted in FIG. 6 includes a portion of an annular processing ring having an outer edge 185 and an inner edge 187. The thermal process chambers 152 can be located within the annular processing ring and, as discussed herein, and may be formed as voids that extend through the body 153, with the covers 182 and 186 defining the volume of the of the thermal process chambers 152 in connection with the voids. To improve compliance or flexibility of the annular processing ring occupied by the process chambers 152, the voids of the thermal process chambers 152 can occupy 50% or more of the volume of the body 153 located within the annular processing ring.

In some embodiments, the inner compression ring (e.g., the inner wall 162 of the cover 160) can contact the sample processing device 150 along the inner edge 187 of the annular processing ring or between the inner edge 187 and the innermost portion of the thermal process chambers 152. Furthermore, in some embodiments, the outer compression ring (e.g., the outer wall 164 of the cover 160) can contact the sample processing device 150 along the outer edge 185 of the annular processing ring or between the outer edge 185 and the outermost portion of the thermal process chambers 152.

Compliance of the annular processing rings in sample processing devices used in connection with the present disclosure can be provided with a combination of an annular processing ring formed as a composite structure using viscoelastic pressure sensitive adhesive and voids located within the annular processing ring. Such a combination may provide more compliance than either approach taken alone.

In the embodiment illustrated in FIGS. 1-6, the sample processing device 150 and the annular cover 160 are each shown as being circular and symmetrical. For example, the annular cover 160 is shown as being a ring-shaped annulus having a symmetrical center 161, such that the inner edge 163 is an inner radial edge 163, and the outer edge 165 is an outer radial edge 165. However, as mentioned above, it should be understood that the annular cover 160 can take on a variety of other suitable shapes. Similarly, the sample processing device 150 can take on a variety of other suitable shapes, and as such, the centers 151 and 161 may not be symmetrical centers, and the inner and outer edges 163 and 165 of the cover 160 may not be “radially” positioned with respect to the center 161. The configuration of the sample processing device 150 and the annular cover 160 shown in FIGS. 1-6 are shown by way of example only.

The annular cover 160 is shown in FIGS. 1-4 and described above as being a separate component from the sample processing device 150. However, it should be understood that, in some embodiments, the annular cover 160 can be integrally formed with the sample processing device 150, and the sample processing device 150 together with the annular cover 160 can together be positioned on the base plate 110.

As mentioned above, in some embodiments, the cover 160 and/or the base plate 110 can include one or more magnetic elements 170 and 172 in the form of electromagnets that can be activated as needed, for example, to provide the compressive force in place of passive magnetic elements. In such an embodiment, electric power can be provided to the electromagnets during rotation of the sample processing device 150.

Although not explicitly depicted in FIGS. 1-3, in some embodiments, the base plate 110 can be constructed such that the thermal structure 130 is exposed on the top first surface 112 as well as on a bottom second surface 114 of the base plate 110. By exposing the thermal structure 130 on the top surface 112 of the base plate 110 (e.g., alone or in addition to the bottom surface 114), a direct thermal path can be provided between the transfer surface 132 of the thermal structure 130 and a sample processing device 150 located between the cover 160 and the base plate 110.

Alternatively or in addition, exposing the thermal structure 130 on the bottom surface 114 of the base plate 110 may provide an advantage when the thermal structure 130 is to be heated by electromagnetic energy emitted by a source directing electromagnetic energy onto the bottom surface 114 of the base plate 110.

By way of example only, the system 100 includes an electromagnetic energy source 190 positioned to deliver thermal energy to the thermal structure 130, with the electromagnetic energy emitted by the source 190 directed onto the bottom surface 114 of the base plate 110 and the portion of the thermal structure 130 exposed on the bottom surface 114 of the base plate 110. Examples of some suitable electromagnetic energy sources may include, but are not limited to, lasers, broadband electromagnetic energy sources (e.g., white light), etc.

While the system 100 is illustrated as including the electromagnetic energy source 190, in some embodiments, the temperature of the thermal structure 130 can be controlled by any suitable energy source that can deliver thermal energy to the thermal structure 130. Examples of potentially suitable energy sources for use in connection with the present disclosure other than electromagnetic energy sources may include, e.g., Peltier elements, electrical resistance heaters, etc.

As used in connection with the present disclosure, the term “electromagnetic energy” (and variations thereof) means electromagnetic energy (regardless of the wavelength/frequency) capable of being delivered from a source to a desired location or material in the absence of physical contact. Nonlimiting examples of electromagnetic energy can include, but are not limited to, laser energy, radio-frequency (RF), microwave radiation, light energy (including the ultraviolet through infrared spectrum), etc. In some embodiments, electromagnetic energy can be limited to energy falling within the spectrum of ultraviolet to infrared radiation (including the visible spectrum).

Where the thermal structure 130 is to be heated by a remote energy source, i.e., an energy source that does not deliver thermal energy to the thermal structure 130 by direct contact, the thermal structure 130 can be constructed to absorb electromagnetic energy and convert the absorbed electromagnetic energy into thermal energy. As a result, the materials used in the thermal structure 130 can possess sufficient thermal conductivity and absorb electromagnetic energy generated by the electromagnetic source 190 at sufficient rates. In addition, it may also be desirable that the material or materials used for the thermal structures 130 have sufficient heat capacity to provide a heat capacitance effect. Examples of some suitable materials include, but are not limited to: aluminum, copper, gold, etc. If the thermal structure 130 is constructed of materials that do not, themselves, absorb electromagnetic energy at a sufficient rate, in some embodiments, the thermal structure 130 can include a material that improves energy absorption. For example, the thermal structure 130 can be coated with an electromagnetic energy absorptive material such as carbon black, polypyrrole, inks, etc.

In addition to selection of suitable materials for the thermal structure 130, it may also be possible to include grooves or other surface structure facing the electromagnetic energy source 190 to increase the amount of surface area exposed to the electromagnetic energy emitted by the source 190. Increasing the surface area of the thermal structure 130 exposed to the electromagnetic energy from source 190 may enhance the rate at which energy is absorbed by the thermal structure 130. The increased surface area used in the thermal structure(s) 130 may also increase the efficiency of electromagnetic energy absorption.

In some embodiments, the thermal structure 130 can be relatively thermally isolated from the remainder of the base plate 110 such that only limited amounts (if any) of the thermal energy in the thermal structure 130 is transferred to the remainder of the base plate 110. That thermal isolation may be achieved, for example, by manufacturing the support structure of the base plate 110 of materials that absorb only limited amounts of thermal energy, e.g. polymers, etc. Some suitable materials for the support structure of base plate 110 include, e.g., glass-filled plastics (e.g., polyetheresterketone), silicones, ceramics, etc.

Although the base plate 110 includes a thermal structure 130 in the form of a substantially continuous circular ring, the thermal structures 130 can alternatively be provided as a series of discontinuous thermal elements, e.g., circles, squares, located beneath the thermal process chambers 152 on the sample processing device 150. One potential advantage, however, of a continuous (e.g., continuous ring) thermal structure 130 is that the temperature of the thermal structure 130 may equilibrate during heating. If a group of thermal process chambers 152 in a sample processing device 150 are arranged such that they are in direct contact with the transfer surface 132 of the thermal structure 130, there is a potential to improve chamber-to-chamber temperature uniformity for all thermal process chambers 152 located above the continuous thermal structure 130.

Although the depicted base plate 110 includes only one thermal structure 130, it will be understood that the base plate can include any number of thermal structures 130 that are necessary to transfer thermal energy to or from the selected thermal process chambers 152 in a sample processing device 150 located thereon. Further, in some embodiments, where more than one thermal structure 130 is provided, the different thermal structures 130 can be independent of each other such that no significant amount of thermal energy is transferred between the different independent thermal structures 130. One example of an alternative in which independent thermal structures 130 are provided may be in the form of concentric annular rings.

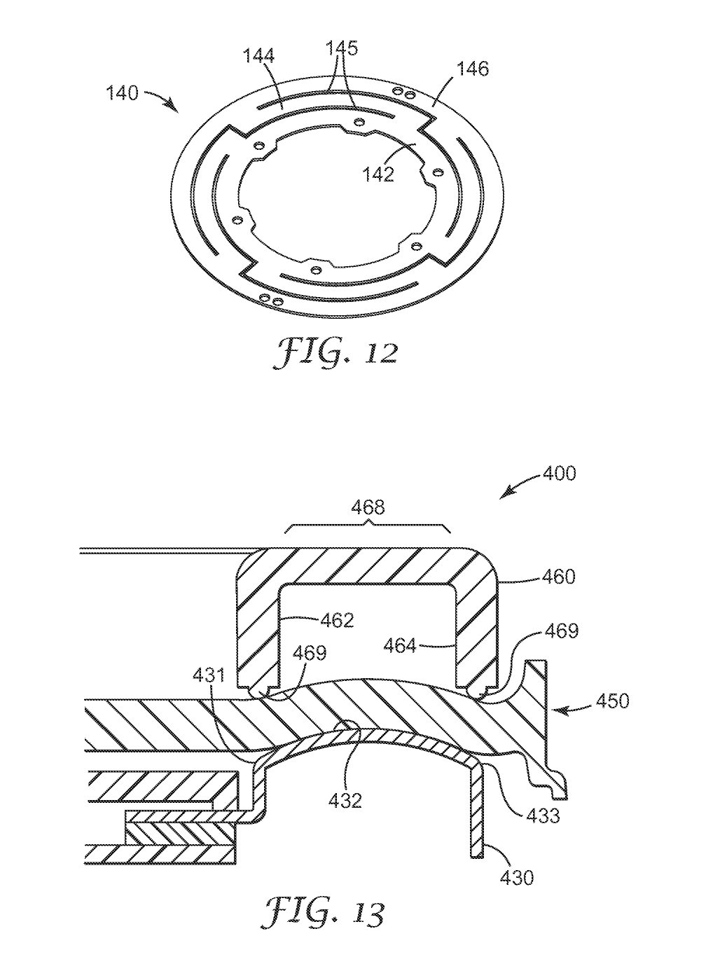

Other features of the system 100 of FIGS. 1-6 are shown in FIGS. 11-12 and described below.

FIGS. 7-8 illustrate another annular compression system 200 according to the present invention, wherein like numerals represent like elements. The system 200 shares many of the same elements and features described above and below with reference to the system 100 of FIGS. 1-6 and 11-12. Accordingly, elements and features corresponding to elements and features in the illustrated embodiment of FIGS. 1-6 and 11-12 are provided with the same reference numerals in the 200 series. Reference is made to the description above or below accompanying FIGS. 1-6 and 11-12 for a more complete description of the features and elements (and alternatives to such features and elements) of the embodiment illustrated in FIGS. 7-8.

The system 200 includes a base plate 210 that rotates about an axis of rotation 211. The base plate 210 can also be attached to a drive system (not shown) in a manner similar to that described above with respect to the system 100, or any suitable alternative arrangement.

As shown in FIGS. 7-8, the system 200 can further include a sample processing device 250 and an annular cover 260 that can be used in connection with the base plate 210. The base plate 210 shown in FIGS. 7-8 is similar to the base plate 110 of the system 100, and includes a thermal structure 230 that can include a thermal transfer surface 232 exposed on a top surface 212 of the base plate 210.

As further shown in FIGS. 7-8, the sample processing device 250 can include thermal process chambers 252 and one or more input wells and/or other chambers (sometimes referred to as “non-thermal” chambers or “non-thermal” process chambers) 254 positioned in fluid communication with the thermal process chambers 252, for example, via one or more channels 258, valves, or the like, or combinations thereof. In addition, the input wells 254 can be positioned between a center 251 of the sample processing device 250 and at least one of the thermal process chambers 252. In addition, similar to the cover 160 described above, the annular cover 260 can be configured to allow access to a portion of the sample processing device 250 that includes the input well(s) 254, such that the input well(s) 254 can be accessed when the cover 260 is positioned adjacent to or coupled to the sample processing device 250.

By way of further example, the sample processing device 250 can include various features and elements, such as those described in PCT Patent Publication No. WO 2008/134470 titled METHODS FOR NUCLEIC ACID AMPLIFICATION (Parthasarathy et al.) and U.S. Patent Publication No. 2008/0152546 titled ENHANCED SAMPLE PROCESSING DEVICES, SYSTEMS AND METHODS (Bedingham et al.).

Similar to the system 100 described above, the annular cover 260 and the base plate 210 can compress a sample processing device 250 located therebetween, for example, to enhance thermal coupling between the thermal structure 230 on the base plate 210 and the sample processing device 250, in addition to holding and/or maintaining the sample processing device 250 on the base plate 210 for rotation about the rotation axis 211. As a result, the rotation axis 211 can define a z-axis of the system 200.

Furthermore, by way of example only and similar to the system 100, the system 200 is depicted in FIGS. 7-8 as including magnetic elements 270 located on (or at least operatively coupled to) the cover 260 and corresponding magnetic elements 272 located on (or at least operatively coupled to) the base plate 210 as an exemplary compression structure.

As shown in FIGS. 7-8, the annular cover 260 can further include a center 261, which can be in line with the rotation axis 211 when the cover 260 is coupled to the base plate 210, an inner edge 263 that at least partially defines an opening 266, and an outer edge 265. As further shown in FIGS. 7-8, the inner edge 263 of the annular cover 260 can be configured to be positioned inwardly (e.g., radially inwardly) of the thermal process chambers 252, relative to the center 261 of the annular cover 260, for example, when the annular cover 260 is positioned adjacent the sample processing device 250. In addition, the inner edge 263 of the annular cover 260 can be configured to be positioned radially outwardly of the input wells 254. Furthermore, the outer edge 265 of the annular cover 260 can be configured to be positioned outwardly (e.g., radially outwardly) of the thermal process chambers 252 (and also outwardly of the input wells 254).

Similar to the system 100, the inner edge 263 can be positioned a first distance d₁′ (e.g., a first radial distance or “first radius”) from the center 261 of the annular cover 260, and the outer edge 265 can be positioned a second distance d₂′ (e.g., a second radial distance or “second radius”) from the center 261 of the annular cover 260. The first distance d₁′ and the second distance d₂′ (and the areas associated with these distances) can have similar relationships as those described above with respect to the system 100.

Similar to the annular cover 160, the annular cover 260 can include an inner wall 262 (e.g., an “inner circumferential wall” or “inner radial wall”; which can function as an inner compression ring, in some embodiments, as described below). As shown, the inner wall 262 can include or define the inner edge 263, and the inner wall 262 can be positioned inwardly (e.g., radially inwardly) of the thermal process chambers 252.

As further shown in FIGS. 7-8, the inner wall 262 can include the magnetic elements 270, such that the magnetic elements 270 form a portion of or are coupled to the inner wall 262. For example, in some embodiments, the magnetic elements 270 can be embedded (e.g., molded) in the inner wall 262. In addition, also similar to the annular cover 160, the annular cover 260 can further include an upper wall 267 that can be positioned to cover a portion of the sample processing device 250, such as a portion that comprises the thermal process chambers 252. In some embodiments, at least a portion of the cover 260, such as one or both of the inner wall 262 and the upper wall 267, can be optically clear.

However, unlike the annular cover 160, the annular cover 260 does not include an outer wall and, as a result, does not provide an outer compression ring to the system 200. Rather, in the system 200, an outer compression ring can be provided by the sample processing device 250.

As shown in FIGS. 7-8, the sample processing device 250 includes an outer wall 255 (or “outer circumferential wall” or “outer radial wall”) that can function as an outer compression ring for compressing at least a portion of the sample processing device 250 onto the thermal transfer surface 232 of the base plate 210. That is, unlike the sample processing device 150 of the system 100, the sample processing device 250 of FIGS. 7-8 includes a taller or thicker outer wall 255 that extends substantially vertically upwardly and contacts the upper wall 267 of the annular cover 260. As a result, in some embodiments, the outer wall 255 can function as an outer compression ring, for example, in conjunction with the upper wall 267, such that the upper wall 267 of the cover 260 can press downwardly (e.g., in a first direction D₁′ along or substantially parallel to the z-axis of the system 200) onto the sample processing device 250, including the outer wall 255 of the sample processing device 250. In some embodiments, the outer wall 255 of the sample processing device 250 can be positioned outwardly (e.g., radially outwardly) of the thermal process chambers 252.

In addition, as shown in FIGS. 7-8, the outer wall 255 of the sample processing device 250 can also function to at least partially isolate the thermal process chambers 252 from ambience and/or from other portions of the sample processing device 250.

Furthermore, by way of example only, as shown in FIGS. 7-8, in some embodiments, the body 253 and/or the outer wall 255 of the sample processing device 250 can include a portion 257 that is adapted to cooperate with the base plate 210. For example, as shown in FIGS. 7-8, the portion 257 of the sample processing device 250 can be dimensioned to receive at least a portion of the base plate 210. Such cooperation between the sample processing device 250 and the base plate 210, for example, can enhance the coupling between the sample processing device 250 and the base plate 210, and can further facilitate the positioning of the sample processing device 250 relative to the base plate 210.

As shown in FIG. 7, the one or more thermal process chambers 252 can be arranged in an annulus about the center 251 of the sample processing device 250, which can sometimes be referred to as an “annular processing ring.” In such embodiments, the annular cover 260 can be adapted to cover and/or isolate a portion of the sample processing device 250 that includes the annular processing ring or the thermal process chambers 252. For example, the annular cover 260 can provide the inner wall 262 and the upper wall 267 to cover and/or isolate the portion of the sample processing device 250 that includes the thermal process chambers 252.

In some embodiments, the sample processing device 250 can include a recess (e.g., an annular recess) 256 formed in the body 253 (e.g., in a top surface of the sample processing device 250) that is dimensioned to receive at least a portion of the annular cover 260. For example, as shown in FIGS. 7-8, the inner wall 262 (including the magnetic elements 270) can be positioned in the recess 256 of the sample processing device 250 when the annular cover 260 is positioned over or coupled to the sample processing device 250.

In addition, as shown in FIGS. 7-8, one or both of the magnetic elements 270 and 272 can be arranged in an annulus, for example, about the rotation axis 211. Furthermore, in some embodiments, at least one of the magnetic elements 270 and 272 can include a substantially uniform distribution of magnetic force about such an annulus.

In some embodiments, the annulus or portion of the cover 260 that includes the magnetic elements 270 can include an inner edge (e.g., an inner radial edge) 273 and an outer edge (e.g., an outer radial edge) 275. As shown in FIGS. 7-8, the cover 260 and/or the magnetic elements 270 can be configured, such that both the inner edge 273 and the outer edge 275 can be positioned inwardly (e.g., radially inwardly) with respect to the thermal process chambers 252.

Furthermore, in some embodiments, the annulus of magnetic elements 270 can be positioned outwardly (e.g., radially outwardly) of the one or more input wells 254, or a portion of the sample processing device 250 (or a portion of the body 253) that includes the input wells 254. In addition, in some embodiments, the input wells 254 (or the portion of the sample processing device 250 that includes or defines the input wells 254) and/or the recess 256 can provide reliable positioning of the cover 260 with respect to the sample processing device 250.

As a result, in some embodiments, the magnetic elements 270 can be restricted to an area of the cover 260 where the magnetic elements 270 are positioned outwardly (e.g., radially outwardly) of the input wells 254 (or other protrusions, chambers, recesses, or formations in the body 253) and inwardly (e.g., radially inwardly) of the thermal process chambers 252. In such configurations, the magnetic elements 270 can be said to be configured to maximize the open area of the sample processing device 250 that is available for access by other devices or for other functions. In addition, in such embodiments, the magnetic elements 270 are not positioned to interrupt or disturb the processing of a sample positioned in the thermal process chambers 252. Furthermore, similar to the system 100, the magnetic elements 270 and 272 can be “keyed” with respect to each other to positioned the cover 260 relative to at least one of the sample processing device 250 and the base plate 210 in a desired orientation.

Similar to the covers 182 and 186 described above with respect to FIGS. 1 and 5, the sample processing device 250 can include a cover 282 that is positioned over a portion of the sample processing device 250 to at least partially define the input wells 254 or other channels, chambers, recesses, etc. of the sample processing device 250.

FIGS. 9-10 illustrate another annular compression system 300 according to the present invention, wherein like numerals represent like elements. The system 300 shares many of the same elements and features described above and below with reference to the system 100 of FIGS. 1-6 and 11-12 or the system 200 of FIGS. 7-8. Accordingly, elements and features corresponding to elements and features in the illustrated embodiment of FIGS. 1-6 and 11-12 or FIGS. 7-8 are provided with the same reference numerals in the 300 series. Reference is made to the description above or below accompanying FIGS. 1-6 and 11-12 and FIGS. 7-8 for a more complete description of the features and elements (and alternatives to such features and elements) of the embodiment illustrated in FIGS. 9-10.

The system 300 includes a cover 360, a sample processing device 350, and a base plate 310. The system 300 is substantially the same as the system 200 of FIGS. 7-8, with the exception that the system 300 includes a cover 360 that does not include an upper wall or an outer wall, but only an inner wall 362. The inner wall 362 comprises one or more magnetic elements 370 adapted to attract one or more magnetic elements 372 in the base plate 310. As a result, at least a portion of the cover 360 can be dimensioned to be received in a recess 356 of the sample processing device 350.

In the embodiment illustrated in FIGS. 9-10, the cover 360 includes a simple annulus that comprises the magnetic elements 370. As shown in FIG. 9, the cover 360 can include an inner edge 363 that defines an opening 366 in the cover 360, and an outer edge 365. In addition, the magnetic elements 370 are shown as being arranged in an annulus that also includes an inner edge 373 and an outer edge 375 (see FIG. 10). In the embodiment illustrated in FIGS. 9 and 10, the inner edge 363 of the cover 360 is spaced a relatively small distance from the inner edge 373 of the magnetic elements 370, and the outer edge 365 of the cover 360 is spaced a relatively small distance from the outer edge 375 of the magnetic elements 370. Said another way, in some embodiments, the inner edge 363 of the cover 360 can be positioned adjacent the inner edge 373 of the magnetic elements 370, and, in some embodiments, the outer edge 365 of the cover 360 can be positioned adjacent the outer edge 375 of the magnetic elements 370. Furthermore, the inner edge 363 of the cover 360, the outer edge 365 of the cover 360, the inner edge 373 of the magnetic elements 370 and the outer edge 375 of the magnetic elements 370 can be positioned inwardly (e.g., radially inwardly) of the thermal process chambers 352, for example, relative to a center 361 of the cover 360 or relative to the rotation axis 311. Other features and elements of the inner and outer edges 363, 373, 365 and 375 (e.g., relative to the thermal process chambers 352), and alternatives thereto, can be found above with respect to the embodiment of FIGS. 1-6 and the embodiment of FIGS. 7-8.

As shown in FIGS. 9 and 10, the cover 360 is not necessarily configured to isolate (e.g., physically or thermally) one or more thermal process chambers 352 in the sample processing device 350 from ambience or from other portions of the sample processing device 350. Rather, the cover 360 is configured to press, hold, and/or deform the sample processing device 350 onto the base plate 310, and particularly, onto a thermal transfer surface 332 of the base plate 310.

Similar to the covers 182 and 186 described above with respect to FIGS. 1 and 5, the sample processing device 350 can include a cover 382 that is positioned over a portion of the sample processing device 350 to at least partially define one or more input wells 354 or other channels, chambers, recesses, etc. of the sample processing device 350. In addition, in some embodiments, the sample processing device 350 can further include an additional cover (not shown) similar to the covers 182 and 186 of FIGS. 1 and 5 positioned over at least a portion of the sample processing device 350 in which the thermal process chambers 352 are formed to at least partially define and/or isolate the thermal process chambers 352.

Returning to the system 100 described above, FIG. 11 is a perspective cross-sectional view of a portion of the base plate 110 and the thermal structure 130 of the system 100 depicted in FIGS. 1-6 taken along line 11-11 in FIG. 1. As shown in FIG. 11, the base plate 110 can include a main body 116 to which the thermal structure 130 is attached. Although not seen in FIG. 11, in some embodiments, the main body 116 can be fixedly attached to a spindle used to rotate the base plate 110. By fixedly attached, it is meant that the main body 116 generally does not move relative to the spindle when a sample processing device 150 is compressed between the cover 160 and the base plate 110 during operation of the system 100.

As depicted in FIG. 11, in some embodiments, the thermal structure 130 can be generally U-shaped below the transfer surface 132. Such shaping can accomplish a number of functions. For example, the U-shaped thermal structure 130 can increase the surface area onto which electromagnetic energy is incident, thus potentially increasing the amount and rate at which energy is transferred to the thermal structure 130. In addition, the U-shaped thermal structure may present a lower thermal mass for the thermal structure 130.

As discussed herein, one optional feature of systems of the present disclosure is the floating or suspended attachment of the thermal structure 130 such that the thermal structure 130 and the cover 160 are resiliently biased towards each other. For example, in some embodiments, the thermal structure 130 can be coupled to the base plate 110 by one or more resilient members, with the one or more resilient members providing a biasing force opposing the force applied by the compression structure (e.g., one or more of the magnetic elements 170 and 172). In some embodiments, the thermal structure 130 can be capable of movement relative to the main body 116 of the base plate 110 in response to compressive forces between the base plate 110 and the cover 160. For example, movement of the thermal structure 130 can be limited to a z-axis direction that can be aligned with (e.g., parallel to) the axis of rotation 111 (e.g., along the first direction D₁).

Resilient coupling of the thermal structure 130 can be advantageous by providing improved compliance with the surface of the sample processing device 150. The floating attachment of the thermal structure 130 can help to compensate for, e.g., surfaces that are not flat, variations in thickness, etc. Resilient coupling of the thermal structure 130 may also improve uniformity in the compressive forces developed between the cover 160 and the thermal structure 130 when a sample processing device 150 is compressed between the two components.

Many different mechanisms can be used to resiliently couple the thermal structure 130. One exemplary mechanism is depicted in FIGS. 11 and 12 in the form of a flat spring 140 that is attached to the main body 116 and the thermal structure 130 of the base plate 110. The depicted flat spring 140 includes an inner ring 142 and spring arms 144 that are at least partially defined by cuts 145 and that extend to an outer ring 146. As shown, the inner ring 142 can be coupled to the main body 116 and the outer ring 146 can be coupled to a flange 136 on the thermal structure 130 (see also FIG. 3). Attachment of the spring 140 can be accomplished by any suitable coupling technique or techniques, e.g., mechanical fasteners, adhesives, solder, brazing, welding, etc.

The forces generated by the flat spring 140 can be adjusted by changing the length of the cuts 145 at least partially defining the spring arms 144, changing the radial width of the spring arms 144, changing the thickness of the spring arms 144 (e.g., in the z-axis direction), selection of materials for the spring 140, etc., or combinations thereof.

In some embodiments, the force urging the base plate 110 and cover 160 towards each other can result in physical contact between the main body 116 of the base plate 110 and the cover 160 within the boundary (e.g., circle) defined by the inner edge of the transfer surface 132 of the thermal structure 130. In other words, the magnetic attraction force in the embodiment shown in FIGS. 1-6 and 11-12 can draw the cover 160 against the main body 116 of the base plate 110. As a result, the forces exerted on the portion of the sample processing device 150 clamped between the cover 160 and the transfer surface 132 can be exerted by the flat spring 140 (or other resilient members if used). In other words, control over the clamping force may be controlled by a resilient member, such as the flat spring 140.

To achieve the result described in the preceding paragraph, in some embodiments, the clamping force can be generated between the cover 160 and the main body 116 of the base plate 110 be greater than the biasing force operating to force the transfer surface 132 of the thermal structure 130 towards the cover 160. As a result, the cover 160 can be drawn into contact with the main body 116, and the resilient member (e.g., the flat spring 40) can control the forces applied to the sample processing device 150 between the cover 160 and the transfer surface 132.

In some embodiments, as shown, an insulating element 138 (see also FIG. 3) can be located between the outer ring 146 of the flat spring 140 and the flange 136 of the base plate 110. The insulating element 138 can serve a number of functions. For example, the insulating element 138 can reduce the transfer of thermal energy between the outer ring 146 of the spring 140 and the flange 136 of the thermal structure 130. Another potential function of the insulating element 138 may be to provide a pre-load to the spring 140, such that the force with which the thermal structure 130 is biased towards the top surface 112 of the base plate 110 is at or above a selected level. A thicker insulating element 138 would typically be expected to increase the pre-load while a thinner insulating element 138 would typically be expected to reduce the pre-load. Examples of some potentially suitable materials for insulating element may include materials with lower thermal conductivity than metals, e.g., polymers, ceramics, elastomers, etc.

Although a flat spring 140 is one example of a resilient member that can be used to resiliently couple the thermal structure 130, many other resilient members could be used in place of or in addition to the depicted flat spring 140. Examples of some other potentially suitable resilient members may include, e.g., leaf springs, elastomeric elements, pneumatic structures (e.g., pistons, bladders, etc.), etc., or combinations thereof.

Although the flat spring 140 and the main body 116 of the base plate 110 are depicted as separate components, alternatives may be possible in which the functions of the main body 116 and the spring 140 are accomplished in a single, unitary component.

FIG. 13 illustrates another annular compression system 400 according to the present invention, wherein like numerals represent like elements. The system 400 shares many of the same elements and features described above with reference to the illustrated embodiment of FIGS. 1-6. Accordingly, elements and features corresponding to elements and features in the illustrated embodiment of FIGS. 1-6 are provided with the same reference numerals in the 400 series. Reference is made to the description above or below accompanying FIGS. 1-6 for a more complete description of the features and elements (and alternatives to such features and elements) of the embodiment illustrated in FIG. 13.

As shown in FIG. 13, the system 400 includes a sample processing device 450 held under compression between a thermal structure 430 of a base plate 410 and a cover 460.

In the embodiment shown in FIG. 13, the transfer surface 432 of the thermal structure 430 can be a shaped surface with a raised portion located between an inner edge 431 and an outer edge 433 (where inner edge 431 is closest to the axis of rotation 411 about which the thermal structure 430 rotates, as discussed herein). The raised portion of the transfer surface 432 can be closer to the cover 460 than the portions of the thermal structure 430 at the inner and outer edges 431 and 433 before the sample processing device 450 is contacted by the cover 460. In some embodiments, as shown in FIG. 13, the transfer surface 432 can have a convex curvature when seen in a radial cross-section. The convex transfer surface 432 may be defined by a circular curve or any other curved profile, e.g., elliptical, etc.

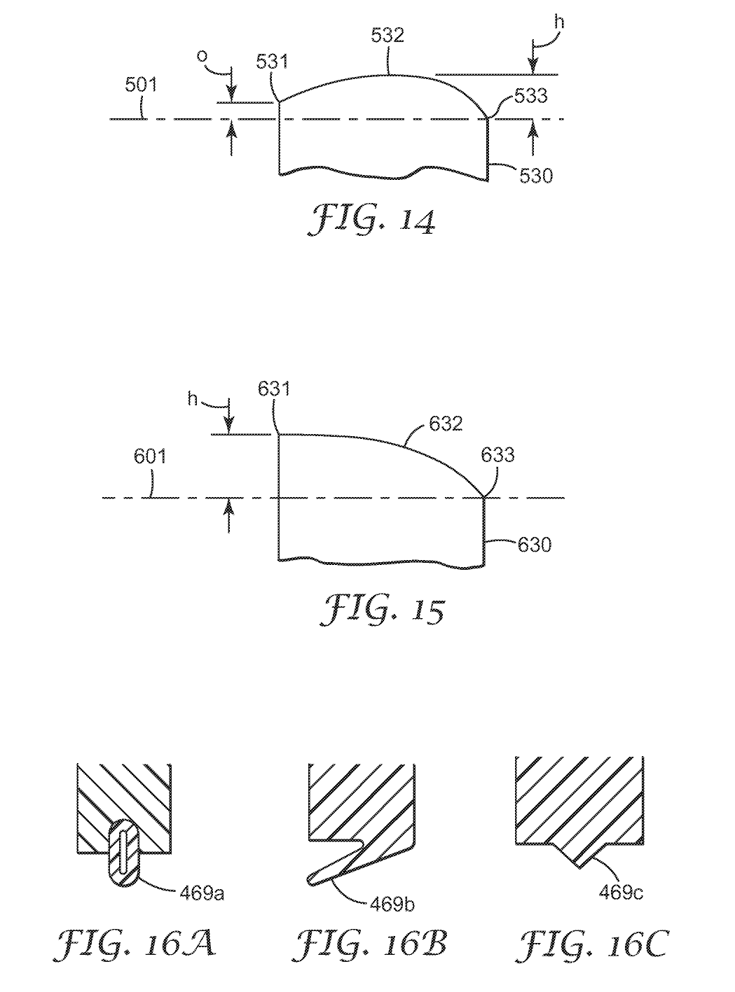

FIGS. 14 and 15 depict alternative shaped transfer surfaces that may be used in connection with thermal structures that are provided as, e.g., annular rings. One such variation as depicted in FIG. 14 includes a thermal structure 530 (depicted in cross-section to illustrate its profile). The thermal structure 530 includes a shaped transfer surface 532 with an inner edge 531 and an outer edge 533. The inner edge 531 is located proximate an axis of rotation about which the thermal structure 530 is rotated as discussed herein. Also depicted is a plane 501 (seen on edge in FIG. 14) that is transverse to the axis of rotation.

In the depicted embodiment, the plane 501 extends through the outer edge 533 of the shaped transfer surface 532. Unlike the transfer surface 432 of FIG. 13 in which the inner and outer edges 431 and 433 are located on the same plane, the inner edge 531 of the transfer surface 532 can be located at an offset (o) distance from the reference plane 501 as depicted in FIG. 14. In some embodiments, as shown, the inner edge 531 of the transfer surface 532 can be located closer to the cover (not shown) than the outer edge 533.

As discussed herein, the shaped transfer surface 532 can include a raised portion between the inner edge 531 and the outer edge 533. The height (h) of the raised portion is depicted in FIG. 14 relative to the plane 501, where the height (h) can represent the maximum height of the raised portion of the transfer surface 532.

Although the shaped transfer surfaces 432 and 532 depicted in FIGS. 12 and 13 include a raised portion with a maximum height located between the inner and outer edges of the transfer surfaces, the maximum height of the raised portion can instead be located at one of the edges of the transfer surface, such as the inner edge. One such embodiment is depicted in FIG. 15 in which a cross-sectional view of a portion of a thermal structure 630 is depicted. The thermal structure 630 includes a shaped transfer surface 632 with an inner edge 631 and an outer edge 633 as discussed above. In some embodiments, the transfer surface 632 can include a raised portion with a height (h) above a reference plane 601 that extends through the outer edge 633 of the transfer surface 632.

Unlike the transfer surfaces of FIGS. 12 and 13, however, the raised portion of the transfer surface 632 has its maximum height (h) located at the inner edge 631. From the maximum height (h), the transfer surface 632 curves downward in a convex curve towards the outer edge 633. In such an embodiment, the inner edge 631 is located at an offset (o) distance from the reference plane 601 that is equal to the height (h).

The amount by which the transfer surfaces 432, 532 deviate from a planar surface may be exaggerated in FIGS. 12-14. The height (h) may in some sense be a function of the radial distance from the inner edge to the outer edge of the transfer surface. In some embodiments, the transfer surface can have a radial width of 4 centimeters or less, in some embodiments, 2 centimeters or less, and in some embodiments, 1 centimeter or less. In such embodiments, the height (h) can be within a range with a lower value greater than zero, such as 0.02 millimeters (mm) or more, and in some embodiments, 0.05 millimeters or more. At the upper end of the range, in some embodiments, the height (h) can be 1 millimeter or less, in some embodiments, 0.5 mm or less, and in some embodiments, 0.25 millimeters or less.

Returning to FIG. 13, by providing a shaped transfer surface in connection with a cover 460 and compression structure of the present disclosure, thermal coupling efficiency between the thermal structure 430 and the sample processing device 450 may be improved. In some embodiments, the shaped transfer surface 432 in combination with the force applied by the cover 460 can deform the sample processing device 450 such that it conforms to the shape of the transfer surface 432. Such deformation of the sample processing device 450 can be useful in promoting contact even if the surface of the sample processing device 450 facing the transfer surface 432 or the transfer surface 432 itself include irregularities that could otherwise interfere with uniform contact in the absence of deformation.

In embodiments in which the sample processing device 450 includes process chambers (see, e.g., thermal process chambers 152 on sample processing device 150 in FIG. 1), the cover 460 can include an optical window 468 that allows for transmission of electromagnetic energy through at least a portion of the cover 460. Such electromagnetic energy may be used to, e.g., monitor process chambers, interrogate process chambers, heat process chambers, move materials in the sample processing device 450, excite materials in the process chambers, etc. By “optical window,” it is meant that the selected portion of the cover 460 transmits electromagnetic energy with selected wavelengths. That transmission may be through transmissive materials (or “optically clear” materials) or through a void formed in the cover 460 (see, e.g., the covers 160, 260 and 360 in FIGS. 1-4, 7-8 and 9-10).

To further promote deformation of the sample processing device 450 to conform to the shape of the transfer surface 432, in some embodiments, the cover 460 can include compression rings 462 and 464 in the cover 460, such that the rings 462 and 464 contact the sample processing device 450—essentially spanning the portion of the sample processing device 450 facing the transfer surface 432. In some embodiments, substantially all compression force transfer between the cover 460 and the thermal structure 430 can occur through the inner and outer compression rings 462 and 464 of the cover 460.

To potentially further enhance conformance of the sample processing device 450 to the transfer surface 432, in some embodiments, the inner and outer compression rings 462 and 464 can include an edge treatment 469 such that minor variations in dimensions of the different components (cover, sample processing device, thermal structure, etc.) can be at least partially compensated for by the edge treatments 469. One example of suitable edge treatments may be a rounded structure that promotes point contact between the sample processing device 450 and the compression rings 462 and 464. Other potential examples of potentially suitable edge treatments may include, e.g., a resilient gasket 469a depicted in FIG. 16A, a cantilevered member 469b depicted in FIG. 16B, and a triangular structure 469c as depicted in FIG. 16C.

In another variation, it should be understood that although the depicted systems include resilient members coupling the thermal structures to the base plates, an alternative arrangement could be used in which the inner and outer compression rings 462 and 464 are resiliently coupled to the cover 460 by one or more resilient members. Resiliently mounting the compression rings 462 and 464 on the cover 460 may also serve to provide some compensation in the system 400 for, e.g., surfaces that are not flat, variations in thickness, etc. Resilient coupling of the compression rings 462 and/or 464 may also improve uniformity in the compressive forces developed between the cover 460 and the thermal structure 430 when a sample processing device 450 is compressed between the two components.

As discussed herein, in some embodiments, the portion of the sample processing device 450 in contact with the transfer surface 432 (or other shaped transfer surfaces) can exhibit some compliance that, under compression, enables the sample processing device 450 to conform to the shape of the transfer surface 432. That compliance may be limited to the portions of the sample processing device located in contact with the transfer surface 432. Some potentially suitable sample processing devices that may include a compliant portion adapted to conform to a shaped thermal transfer surface are described in, e.g., U.S. Patent Publication No. 2007/0009391 titled COMPLIANT MICROFLUIDIC SAMPLE PROCESSING DISKS (Bedingham et al.) and U.S. Patent Publication No. 2008/0050276 titled MODULAR SAMPLE PROCESSING APPARATUS KITS AND MODULES (Bedingham et al.).

One embodiment of the present disclosure includes a system for processing sample processing devices, the system comprising: a base plate operatively coupled to a drive system, wherein the drive system rotates the base plate about a rotation axis, and wherein the rotation axis defines a z-axis; a thermal structure operatively coupled to the base plate, wherein the thermal structure comprises a transfer surface exposed proximate a first surface of the base plate; at least one first magnetic element operatively coupled to the base plate; a sample processing device comprising at least one thermal process chamber; an annular cover adapted to face the transfer surface, the annular cover having a center, an inner edge, and an outer edge, the sample processing device adapted to be positioned between the base plate and the annular cover, the inner edge of the annular cover configured to be positioned inwardly of the at least one thermal process chamber, relative to the center of the annular cover, when the sample processing device is positioned adjacent the annular cover; and at least one second magnetic element operatively coupled to the annular cover, the at least one second magnetic element configured to attract the at least one first magnetic element to force the annular cover in a first direction along the z-axis, such that at least a portion of the sample processing device is urged into contact with the transfer surface of the base plate.

Another embodiment of the present disclosure includes a system for processing sample processing devices, the system comprising: a base plate operatively coupled to a drive system, wherein the drive system rotates the base plate about a rotation axis, and wherein the rotation axis defines a z-axis; a thermal structure operatively coupled to the base plate, wherein the thermal structure comprises a transfer surface exposed proximate a first surface of the base plate; a first annulus of magnetic elements operatively coupled to the base plate; a sample processing device comprising at least one thermal process chamber; an annular cover adapted to face the transfer surface, the annular cover having an inner edge and an outer edge, the inner edge being positioned inwardly of the at least one thermal process chamber, the sample processing device adapted to be positioned between the base plate and the annular cover; and a second annulus of magnetic elements operatively coupled to the annular cover, the second annulus of magnetic elements configured to attract the first annulus of magnetic elements to force the annular cover in a first direction along the z-axis, such that at least a portion of the sample processing device is urged into contact with the transfer surface of the base plate.

Another embodiment of the present disclosure includes a method for processing sample processing devices, the method comprising: providing a base plate operatively coupled to a drive system; providing a thermal structure operatively coupled to the base plate, wherein the thermal structure comprises a transfer surface exposed proximate a first surface of the base plate; providing a sample processing device comprising at least one thermal process chamber; providing an annular cover facing the transfer surface, the annular cover having an inner edge and an outer edge; providing at least one first magnetic element operatively coupled to the base plate and at least one second magnetic element operatively coupled to the annular cover; positioning the sample processing device between the base plate and the annular cover, such that the inner edge of the annular cover is positioned inwardly of the at least one thermal process chamber, and such that the at least one first magnetic element attracts the at least one second magnetic element to force the annular cover in a first direction along the z-axis, such that at least a portion of the sample processing device is urged into contact with the transfer surface of the base plate; and rotating the base plate about a rotation axis, wherein the rotation axis defines a z-axis.

In any of the embodiments above, the sample processing device can further comprise at least one non-thermal process chamber positioned inwardly of the inner edge of the annular cover when the sample processing device is positioned adjacent the annular cover.

In any of the embodiments above, the inner edge of the annular cover can include an inner radial edge, and the inner radial edge can be positioned radially inwardly of the at least one thermal process chamber.

In any of the embodiments above, the outer edge of the annular cover can include an outer radial edge.

In any of the embodiments above, the at least a portion of the sample processing device can include the at least one thermal process chamber.

In any of the embodiments above, the sample processing device can include a recess, and the annular cover can include a portion dimensioned to be received in the recess of the sample processing device.

In any of the embodiments above, the at least one thermal process chamber can be arranged in an annulus about the rotation axis.

In any of the embodiments above, the at least one thermal process chamber can be arranged within an annular processing ring, and the at least a portion of the sample processing device can include the annular processing ring.

In any of the embodiments above, the outer edge of the annular cover can be positioned inwardly of the at least one thermal process chamber.

In any of the embodiments above, the outer edge of the annular cover can be positioned outwardly of the at least one thermal process chamber.

In any of the embodiments above, the annular cover can include a wall adapted to be positioned over the at least one thermal process chamber. In some embodiments, the wall can be optically clear.

In any of the embodiments above, at least a portion of the annular cover can be optically clear.

In any of the embodiments above, at least one of the annular cover and the sample processing device can include an outer wall that is positioned outwardly of the at least one thermal process chamber to thermally isolate the at least one thermal process chamber.

In any of the embodiments above, the inner edge can be an inner radial edge positioned a first radial distance from a center of the annular cover, and the outer edge can be an outer radial edge positioned a second radial distance from the center of the annular cover.

In any of the embodiments above, the first radial distance can be at least about 50% of the second radial distance.

In any of the embodiments above, the annular cover can include an opening positioned to provide access to the sample processing device.

In any of the embodiments above, the outer edge of the annular cover can be positioned a first radius from a center of the annular cover, and the first radius can define a first area. In such embodiments, the area of the opening can be at least 30% of the first area.

In any of the embodiments above, the sample processing device can include at least one input well adapted to be in fluid communication with at least one of the at least one thermal process chamber, and the at least one input well can be further positioned between a center of the sample processing device and at least one of the at least one thermal process chamber.

In any of the embodiments above, the annular cover can be adapted to allow access to at least one of the at least one input well when the sample processing device is positioned adjacent the annular cover.

In any of the embodiments above, the annular cover can include an opening positioned to provide access to at least one of the at least one input well when the sample processing device is positioned adjacent the annular cover.

In any of the embodiments above, the annular cover can include a portion that covers at least one of the at least one thermal process chamber when the sample processing device is positioned adjacent the annular cover.

In any of the embodiments above, the annular cover can be integrally formed with the sample processing device.

In any of the embodiments above, at least one of the at least one first magnetic element and the at least one second magnetic element can include a ferromagnetic material.

In any of the embodiments above, the at least one second magnetic element can include an inner edge and an outer edge, and both the inner edge and the outer edge can be positioned inwardly of the at least one thermal process chamber.

In any of the embodiments above, the annular cover can include an inner wall comprising the at least one second magnetic element and an outer wall positioned outwardly of the at least one thermal process chamber when the sample processing device is positioned adjacent the annular cover.

In any of the embodiments above, the at least one first magnetic element and the at least one second magnetic element can be keyed with respect to each other, such that the annular cover and the base plate can be adapted to be positioned in a prescribed orientation with respect to each other.

In any of the embodiments above, at least one of the at least one first magnetic element and the at least one second magnetic element can be in the form of an annulus, positioned about the rotation axis.

In any of the embodiments above, at least one of the at least one first magnetic element and the at least one second magnetic element can include a substantially uniform distribution of magnetic force about the annulus.

In any of the embodiments above, the at least one second magnetic element can be arranged in the form of an annulus about the rotation axis, and the annulus can include an outer edge. In such embodiments, the outer edge of the annular cover can be positioned adjacent the outer edge of the annulus.

In any of the embodiments above, the at least one second magnetic element can be arranged in the form of an annulus about the rotation axis, the annulus can include an outer edge, and the outer edge can be positioned inwardly of the at least one thermal process chamber, for example, when the sample processing device is positioned adjacent the annular cover.

In any of the embodiments above, the second annulus of magnetic elements can include an inner edge and an outer edge, and both the inner edge and the outer edge can be positioned inwardly of the at least one thermal process chamber.

In any of the embodiments above, the annular cover can include an inner wall comprising the second annulus of magnetic elements and an outer wall positioned outwardly of the at least one thermal process chamber when the sample processing device is positioned adjacent the annular cover.

In any of the embodiments above, the first annulus of magnetic elements and the second annulus of magnetic elements can be keyed with respect to each other, such that the annular cover and the base plate are adapted to be positioned in a prescribed orientation.

In any of the embodiments above, at least one of the first annulus of magnetic elements and the second annulus of magnetic elements can include a substantially uniform distribution of magnetic force about the annulus.

In any of the embodiments above, the second annulus of magnetic elements can include an outer edge, and the outer edge of the annular cover can be positioned adjacent the outer edge of the second annulus of magnetic elements.

In any of the embodiments above, the second annulus of magnetic elements can include an outer edge, and the outer edge can be positioned inwardly of the at least one thermal process chamber when the sample processing device is positioned adjacent the annular cover.

In any of the embodiments above, the inner edge of the annular cover can define an opening, and any of the method embodiments above can further include accessing at least a portion of the sample processing device via the opening in the annular cover, wherein accessing can include at least one of physically accessing, optically accessing, and thermally accessing at least a portion of the sample processing device.

While various embodiments of the present disclosure are shown in the accompanying drawings by way of example only, it should be understood that a variety of combinations of the embodiments described and illustrated herein can be employed without departing from the scope of the present disclosure. For example, some embodiments of the system of the present disclosure can include a base plate from one embodiment, a sample processing device from another embodiment, and a cover from another embodiment.

In addition, the embodiments described above and illustrated in the figures are presented by way of example only and are not intended as a limitation upon the concepts and principles of the present disclosure. As such, it will be appreciated by one having ordinary skill in the art that various changes in the elements and their configuration and arrangement are possible without departing from the spirit and scope of the present disclosure.

All references and publications cited herein are expressly incorporated herein by reference in their entirety into this disclosure.

Various features and aspects of the present disclosure are set forth in the following claims.

What is claimed is:

1. A system for processing sample processing devices, the system comprising:
a base plate operatively coupled to a drive system, wherein the drive system rotates the base plate about a rotation axis, and wherein the rotation axis defines a z-axis;
a thermal structure operatively coupled to the base plate, wherein the thermal structure comprises a transfer surface exposed proximate a first surface of the base plate;
at least one first magnetic element operatively coupled to the base plate;
a sample processing device comprising at least one thermal process chamber;
an annular cover adapted to face the transfer surface, the annular cover having a center, an inner edge, and an outer edge, the sample processing device adapted to be positioned between the base plate and the annular cover, the inner edge of the annular cover configured to be positioned inwardly of the at least one thermal process chamber, relative to the center of the annular cover, when the sample processing device is positioned adjacent the annular cover; and
at least one second magnetic element operatively coupled to the annular cover, the at least one second magnetic element configured to attract the at least one first magnetic element to force the annular cover in a first direction along the z-axis, such that at least a portion of the sample processing device is urged into contact with the transfer surface of the base plate.

2. The system of claim 1, wherein the sample processing device further comprises at least one non-thermal process chamber positioned inwardly of the inner edge of the annular cover when the sample processing device is positioned adjacent the annular cover.

3. The system of claim 1, wherein the inner edge of the annular cover includes an inner radial edge, and wherein the inner radial edge is positioned radially inwardly of the at least one thermal process chamber.

4. The system of claim 1, wherein the outer edge of the annular cover includes an outer radial edge.

5. The system of claim 1, wherein the at least a portion of the sample processing device includes the at least one thermal process chamber.

6. The system of claim 1, wherein the sample processing device includes a recess, and wherein the annular cover includes a portion dimensioned to be received in the recess of the sample processing device.

7. The system of claim 1, wherein the at least one thermal process chamber is arranged within an annular processing ring, and wherein the at least a portion of the sample processing device includes the annular processing ring.

8. The system of claim 1, wherein the outer edge of the annular cover is positioned inwardly of the at least one thermal process chamber.

9. The system of claim 1, wherein the outer edge of the annular cover is positioned outwardly of the at least one thermal process chamber.

10. The system of claim 1, wherein the annular cover includes a wall adapted to be positioned over the at least one thermal process chamber.

11. The system of claim 1, wherein at least a portion of the annular cover is optically clear.

12. The system of claim 1, wherein at least one of the annular cover and the sample processing device includes an outer wall that is positioned outwardly of the at least one thermal process chamber to thermally isolate the at least one thermal process chamber.

13. The system of claim 1, wherein the inner edge is an inner radial edge positioned a first radial distance from a center of the annular cover, and wherein the outer edge is an outer radial edge positioned a second radial distance from the center of the annular cover.

14. The system or of claim 13, wherein the first radial distance is at least about 50% of the second radial distance.

15. The system of claim 1, wherein the annular cover includes an opening positioned to provide access to the sample processing device.

16. The system of claim 15, wherein the outer edge of the annular cover is positioned a first radius from a center of the annular cover, wherein the first radius defines a first area, and wherein the area of the opening is at least 30% of the first area.

17. The system of claim 1, wherein the sample processing device includes at least one input well adapted to be in fluid communication with at least one of the at least one thermal process chamber, the at least one input well further positioned between a center of the sample processing device and at least one of the at least one thermal process chamber.

18. The system of claim 17, wherein the annular cover is adapted to allow access to at least one of the at least one input well when the sample processing device is positioned adjacent the annular cover.

19. The system of claim 1, wherein the annular cover is integrally formed with the sample processing device.

20. The system of claim 1, wherein the at least one second magnetic element includes an inner edge and an outer edge, and wherein both the inner edge and the outer edge are positioned inwardly of the at least one thermal process chamber.

21. The system of claim 1, wherein the annular cover includes an inner wall comprising the at least one second magnetic element and an outer wall positioned outwardly of the at least one thermal process chamber when the sample processing device is positioned adjacent the annular cover.

22. The system of claim 1, wherein the at least one first magnetic element and the at least one second magnetic element are keyed with respect to each other, such that the annular cover and the base plate are adapted to be positioned in a prescribed orientation with respect to each other.

23. The system of claim 1, wherein at least one of the at least one first magnetic element and the at least one second magnetic element is in the form of an annulus, positioned about the rotation axis.

24. The system of claim 1, wherein the at least one second magnetic element is arranged in the form of an annulus about the rotation axis, wherein the annulus includes an outer edge, and wherein the outer edge is positioned inwardly of the at least one thermal process chamber when the sample processing device is positioned adjacent the annular cover.

25. A method for processing sample processing devices, the method comprising:
providing a base plate operatively coupled to a drive system;
providing a thermal structure operatively coupled to the base plate, wherein the thermal structure comprises a transfer surface exposed proximate a first surface of the base plate;
providing a sample processing device comprising at least one thermal process chamber;
providing an annular cover facing the transfer surface, the annular cover having an inner edge and an outer edge;
providing at least one first magnetic element operatively coupled to the base plate and at least one second magnetic element operatively coupled to the annular cover;
positioning the sample processing device between the base plate and the annular cover, such that the inner edge of the annular cover is positioned inwardly of the at least one thermal process chamber, and such that the at least one first magnetic element attracts the at least one second magnetic element to force the annular cover in a first direction along the z-axis, such that at least a portion of the sample processing device is urged into contact with the transfer surface of the base plate; and
rotating the base plate about a rotation axis, wherein the rotation axis defines a z-axis.

26. The method of claim 25, wherein the inner edge of the annular cover defines an opening, and further comprising accessing at least a portion of the sample processing device via the opening in the annular cover.

27. The method of claim 26, wherein accessing includes at least one of physically accessing, optically accessing, and thermally accessing at least a portion of the sample processing device.
